(19) United States
(12) Patent Application Publication
(10) Pub. No.: US 2023/0390386 A1
(43) Pub. Date: Dec. 7, 2023

(54) REPLICATION-DEFECTIVE VACCINES AND USES THEREOF

(71) Applicant: Rutgers, The State University of New Jersey, New Brunswick, NJ (US)

(72) Inventors: Hua ZHU, New Brunswick, NJ (US); Dabbu Kumar Jaijyan, New Brunswick, NJ (US); Moses Lee, Vancouver, WA (US)

(21) Appl. No.: 18/250,779

(22) PCT Filed: Oct. 26, 2021
(86) PCT No.: PCT/US2021/056531

§ 371 Date: May 5, 2023

Related U.S. Application Data
(60) Provisional application No. 63/106,227, filed on Oct. 27, 2020.

(51) Int. Cl. A61K 39/25; A61P 37/02

(52) U.S. Cl. CPC A61K 39/25; A61P 37/02; A61K 2039/522

(57) ABSTRACT

The present invention features a method of producing an immunogenic composition against a pathogen, wherein the pathogen is a virus or bacterium. Also provided is an immunogenic composition produced thereof, as well as methods of use comprising said immunogenic composition.

### CROSS-REFERENCE TO RELATED APPLICATION

The present application claims priority under 35 U.S.C. § 119(e) to U.S. Provisional Patent Application No. 63/106,227, filed Oct. 27, 2020, which is hereby incorporated by reference in its entirety herein.

### BACKGROUND OF THE INVENTION

DNA viruses are responsible for thousands of infected cases and mortality around the world. There are 3 orders and 31 families of DNA viruses, with wide host range including invertebrate, vertebrates, plants, marine organisms, and others. There may be many other unidentified DNA viruses, any of which may cause severe diseases.

Newly emerged DNA viruses may cause severe infections as they are not associated with herd immunity within the infected population. DNA viruses that belong to the papovavirus, hepadnavirus, herpesvirus, and adenovirus families can cause tumor formation in humans and animals. More specifically, DNA viruses such as Epstein Barr Virus (EBV), Human papillomavirus (several forms of cancer), Kaposi's sarcoma associated virus (skin cancer), human cytomegalovirus (many form of cancer), Hepatitis B virus (Hepatocarcinoma) and Markel cell polyomavirus (Merkel cell carcinoma) are classified as tumor viruses. Moreover, DNA viruses are the causative agent of diseases including genital herpes, chickenpox, CMV retinitis, roseola, conjunctivitis, pneumonia, cancers, congenital disease, and many others. Antiviral therapies for the human simplex virus, cytomegalovirus, and varicella-zoster virus reduce the cases significantly but other viruses with limited antiviral therapy continue to cause morbidity and mortalities.

Generation of replication-defective and live-attenuated DNA viruses and bacteria represent a promising immunization strategy to combat many viral and bacterial diseases affecting human health. Replication-defective viruses lack the ability to replicate their genomes, and are thus unable to produce infectious progeny virus in infected cells; as a result, they are restricted majorly to the site of inoculation. Likewise, replication-defective bacterial cells retain their metabolically activity without the ability to reproduce and generate pathogenic infection. Vaccines produced from replication-defective viruses and bacteria have many advantages over the classical inactivated or subunit vaccines, as they are safe, inactive, and present antigens in a context more closely resembling the pathogenic cells and bacteria they are derived from. It has been reported that a subunit vaccine does not induce enough memory T cells to provide complete protection against many diseases and there is a need to unravel new strategies to effectively eradicate the viral diseases. A replication-defective viral vaccine has been tested for many viruses such as human simplex virus-1, human simplex virus-2, smallpox candidate vaccine, and influenza virus-like particles. Recently, Merck has tested a replication-defective virus vaccine for cytomegalovirus in phase 2 clinical trial that shows protection.

Thus, there is a need for rapid and broadly-applicable methods of generating vaccines and other immunogenic compositions against bacteria and DNA viruses in both humans and other mammals, as well as new vaccines that induce optimal, protective immune responses in subjects. The current invention addresses these needs.

### SUMMARY OF THE INVENTION

As described herein, the present invention relates in part to methods of producing immunogenic compositions comprising bacterial cells and DNA virus particles that have been rendered viable but replication-deficient via treatment with centanamycin, tafuramycin, duocarmycin, or related compounds, as well as to such immunogenic compositions. Also included are methods of using said immunogenic compositions as vaccines to immunize subjects against the bacterial cells or viruses or combinations thereof.

As such in one aspect, the invention provides a method of producing an immunogenic composition against a pathogen, wherein the pathogen is a virus or bacterium. In certain embodiments, the method comprises providing isolated or purified particles and/or cells of the pathogen, or infected cells comprising the pathogen. In certain embodiments, the method comprises contacting the pathogenic particles and/or cells, or infected cells comprising the pathogen, with centanamycin, or a salt, solvate, analog, or derivative thereof, thereby rendering the pathogenic particles or cells attenuated and replication-defective. In certain embodiments, the method comprises isolating the attenuated and replication-defective pathogenic particles or cells, thereby producing the immunogenic composition.

In certain embodiments, the pathogen is a virus.

In certain embodiments, the virus is a DNA virus.

In certain embodiments, the DNA virus is selected from the group consisting of adenoviridae, papovaviridae, parvoviridaem, herpesviridae, poxviridae, anelloviridae, pleolipoviridae, and any combination thereof.

In certain embodiments, the pathogen is a bacterium.

In certain embodiments, the bacterium is selected from the group consisting of Escherichia coli, a Mycobacterium, a Streptococcus, a Pseudomonas, a Staphylococcus, a Chlamydia, a Neisseria, Borrelia burgdorferi, a Bruccella, a Listeria, a Legionella, a Shigella, a Campylobacter, a Salmonella, and any combination thereof.

In certain embodiments of the above aspects or any aspect or embodiment provided herein, the pathogen comprises one or more bacteria and/or one or more viruses.

In another aspect, the invention provides an immunogenic composition comprising a pharmaceutically acceptable carrier or diluent and an effective amount of isolated or purified particles and/or cells from a pathogen, wherein the pathogen is a virus or bacterium, wherein the isolated or purified pathogenic particles and/or cells are attenuated and replication-defective due to treatment with a compound selected from centanamycin or tafuramycin A, or a salt, solvate, analog, or derivative thereof, and wherein the composition elicits a protective immune response to the pathogenic particles and/or cells in a subject.

In certain embodiments, the compound is a compound of Formula (I):

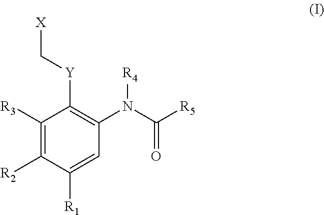

wherein,

R₁ is NHR or OR, where R is selected from H, benzyl, benzyloxycarbonyl, 4-nitrobenzyloxycarbonyl and N′-methylpiperazinyl-N-carbonyl;

R₂ is selected from H and C₁₋₆ alkyl;

R₃ is selected from H and C₁₋₆ alkyl;

or R₂ and R₃, form a fused ring selected from the group consisting of benzene, pyrrole, pyridine, furan, and 5-methylfuran, wherein the fused ring may be optionally substituted with C₁₋₆ alkyl, CF₃, or C₁₋₆ alkyloxycarbonyl;

Y is —CH— to form a five-membered ring with R₄;

X is an electrophilic leaving group;

R₄ is a —CH₂— group bonded to Y to form a five-membered ring; and

R₅ is selected from the group consisting of:

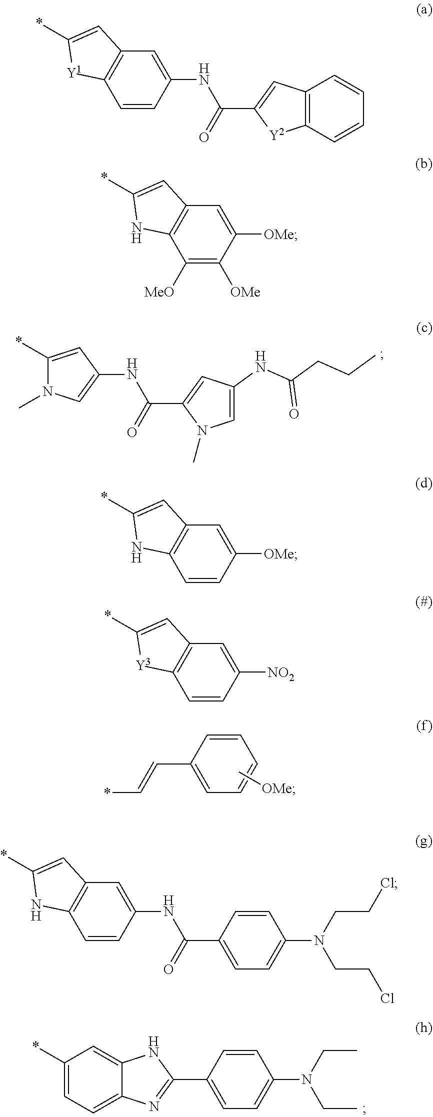

wherein Y¹ and Y² are independently selected from O and NH and wherein Y³ is independently selected from O and NH.

In certain embodiments, the compound is a compound of Formula (II):

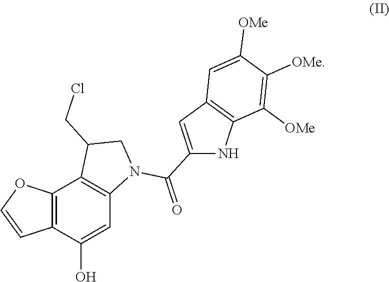 [SMILES: COc1cc2cc(C(=O)N3CC(CCl)c4c3cc(O)c3ccoc43)[nH]c2c(OC)c1OC]

In certain embodiments, the compound is a compound of Formula (III):

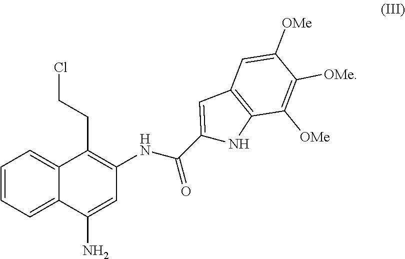 [SMILES: COc1cc2cc(C(=O)Nc3cc(N)c4ccccc4c3CCCl)[nH]c2c(OC)c1OC]

In certain embodiments, the compound is a compound of Formula (IV):

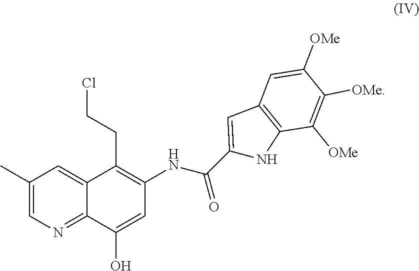 [SMILES: COc1cc2cc(C(=O)Nc3cc(O)c4ncc(C)cc4c3CCCl)[nH]c2c(OC)c1OC]

In certain embodiments, the pathogen is a virus.

In certain embodiments, the virus comprises a DNA virus.

In certain embodiments, the DNA virus is selected from the group consisting of adenoviridae, papovaviridae, parvoviridaem, herpesviridae, poxviridae, anelloviridae, pleolipoviridae, and any combination thereof.

In certain embodiments, the pathogen is a bacterium.

In certain embodiments, the bacterium is selected from the group consisting of Escherichia coli, a Mycobacterium, a Streptococcus, a Pseudomonas, a Staphylococcus, a Chlamydia, a Neisseria, Borrelia burgdorferi, a Bruccella, a Listeria, a Legionella, a Shigella, a Campylobacter, a Salmonella, and any combination thereof.

In certain embodiments of the above aspects or any other aspect or embodiment provided herein, the pathogen comprises one or more bacteria and/or one or more viruses.

In another aspect, the invention provides a method of stimulating an immune response against a pathogen in a subject, wherein the pathogen is a virus or bacterium, the method comprising administering to the subject an effective amount of the immunogenic composition of any one of the above aspects or any other aspect or embodiment provided herein.

In certain embodiments, the subject is a mammal.

In certain embodiments, the subject is human.

In another aspect, the invention provides a method for treating, ameliorating, and/or preventing a pathogen-caused disease in a subject, wherein the pathogen is a virus or bacterium, the method comprising administering to the subject an effective amount of the immunogenic composition of any one of the above aspects or any aspect or embodiment provided herein.

In certain embodiments, the disease is a viral disease.

In certain embodiments, the viral disease is caused by a DNA virus.

In certain embodiments, the DNA virus selected from the group consisting of adenoviridae, papovaviridae, parvoviridaem, herpesviridae, poxviridae, anelloviridae, pleolipoviridae, and any combination thereof.

In certain embodiments, the DNA virus is a Herpesviridae.

In certain embodiments, the DNA virus is varicella-zoster virus (VZV).

In certain embodiments, the DNA virus is cytomegalovirus (CMV).

In certain embodiments, the disease is a bacterial disease.

In certain embodiments, the bacterial disease is caused by a bacterium selected from the group consisting of Escherichia coli, a Mycobacterium, a Streptococcus, a Pseudomonas, a Staphylococcus, a Chlamydia, a Neisseria, Borrelia burgdorferi, a Bruccella, a Listeria, a Legionella, a Shigella, a Campylobacter, a Salmonella, and any combination thereof.

In certain embodiments, the subject is a mammal.

In certain embodiments, the subject is human.

### BRIEF DESCRIPTION OF THE DRAWINGS

The following detailed description of selected embodiments of the invention will be better understood when read in conjunction with the appended drawings. For the purpose of illustrating the invention, selected embodiments are shown in the drawings. It should be understood, however, that the invention is not limited to the precise arrangements and instrumentalities of the embodiments shown in the drawings.

FIGS. 1A-1C illustrate that centanamycin (CM) inhibits the growth of the Toledo-Luc strain of human cytomegalovirus (HCMV) and AD169 GFP cells. FIG. 1A: In vitro growth curve analysis of Toledo-Luc treated with different concentration of CM using MRC5 cells. FIG. 1B: Ex vivo growth curve analysis of Toledo-Luc treated with different concentration of CM in fetal thymus tissue. Error bars represent the standard deviation in each figure. FIG. 1C: Fluorescence image of AD169-GFP treated with different concentration of centanamycin (100 μM, 10 μM, 1 μM and 0.1 μM)

FIGS. 2A-2B illustrate that centanamycin inhibits growth of HSV-2. FIG. 2A: Fluorescence image of HSV-2 GFP treated with different concentrations of centanamycin. FIG. 2B depicts growth curve analysis of HSV-2 GFP in presence of different concentrations of centanamycin. Error bars corresponds to standard deviation.

FIGS. 3A-3C illustrate a non-limiting analysis of virus DNA after centanamycin treatment. FIG. 3A depicts virus DNA treatment with and without CM treatment. FIG. 3B depicts HCMV gDNA treated with 10 μM of centanamycin digested with EcoR1. FIG. 3C depicts HCMV DNA treated with CM and incubated at 50° C. for 30 minutes to check thermolabile methylation by centanamycin.

FIGS. 4A-4F illustrate non-limiting in vivo protection of CM treated murine cytomegalovirus (MCMV). FIG. 4A depicts a growth curve analysis of MCMV-Luc Growth curve analysis of MCMV-Luc in presence of centanamycin. FIG. 4B: In vivo analysis of CM treated MCMV-Luc. FIG. 4C depicts a strategy for mice immunization. FIG. 4D illustrates antibody titer determination in the CM treated MCMV immunized mice. FIG. 4E depicts an antibody neutralization assay for MCMV. FIG. 4F depicts a determination of viral titer in challenged mice.

FIG. 5 illustrates a non-limiting effect of centanamycin on Varicella zoster virus expressing luciferase (VZV-Luc) growth. VZV-Luc growth curve analysis in presence of different concentration of CM.

FIGS. 6A-6B illustrate a non-limiting treatment of E. coli with centanamycin. FIG. 6A is a table illustrating the effect of centanamycin treatment on bacterial growth on agar plates after 16 h of incubation. FIG. 6B illustrates that centanamycin treatment of E. coli reduces the bacterial load.

FIG. 7 illustrates a non-limiting effect of centanamycin on the growth of E. coli bacteria.

FIG. 8 is a non-limiting table of DNA viruses contemplated for targeting by the methods of the invention.

FIGS. 9A-9D are non-limiting tables of bacteria contemplated for targeting by the methods of the invention.

FIGS. 10A-10B illustrate the effect of centanamycin treatment on the growth of VZV-Luc. FIG. 10A is an in vivo growth curve analysis of VZV treated with different concentrations of CM. Acyclovir (30 mg/kg) was used as a positive control. Each data point represents the average of triplicate and error bars show the standard deviation. FIG. 10B shows a representative image of a mouse in the treatment group. VZV-Luc represents one of the mice treated with vehicle only (PET).

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or within 10%, 9%, 8%, 7%, 6%, 5%, 4%, 3%, 2%, 1%, 0.5%, 0.1%, 0.05%, or 0.01% of the specified value, as such variations are appropriate to perform the disclosed methods. Unless otherwise clear from context, all numerical values provided herein are modified by the term about.

By “ameliorate” is meant decrease, suppress, attenuate, diminish, arrest, or stabilize the development or progression of a disease. An exemplary disease is a bacterial or viral infection, and associated symptoms.

As used herein, the term “antibody” means whole, intact antibody molecules, as well as fragments of antibody molecules that retain immunogen-binding ability, including the well-known active fragments F(ab′)2, and Fab. The antibodies of the invention comprise whole native antibodies, bispecific antibodies; chimeric antibodies; Fab, Fab′, single chain V region fragments (scFv), fusion polypeptides, and unconventional antibodies.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “binding to” a molecule is meant having a physicochemical affinity for that molecule.

By the term “specifically binds,” as used herein, is meant a ligand, which recognizes and binds with a binding partner present in a sample, but which ligand does not substantially recognize or bind other polypeptides in the sample.

By “decreases” is meant a negative alteration of at least 10%, 25%, 50%, 75%, 100%, 200%, 300%, 400%, 500%, 1000%, or more.

By “effective amount of” is meant an amount of an immunogenic composition sufficient to induce or enhance an immune response in a subject. Levels of induced immunity can be monitored, e.g., by measuring amounts of neutralizing secretory and/or serum antibodies, e.g., by enzyme-linked immunosorbent assay, agglutination assay or any other method known in the art. The effective amount of active compound(s) used to practice the present invention for prophylaxis or for therapeutic treatment of a disease varies depends upon the manner of administration, the age, body weight, and general health of the subject. Ultimately, the attending physician or veterinarian will decide the appropriate amount and dosage regimen. Such amount is referred to as an “effective” amount.

The term “expression” as used herein is defined as the transcription and/or translation of a particular nucleotide sequence driven by its promoter.

“Isolated” means altered or removed from the natural state or otherwise been subjected to human manipulation. For example, a nucleic acid or a peptide naturally present in a living organism is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Isolated material may be substantially or essentially free from components that normally accompany it in its natural state, or may be manipulated so as to be in an artificial state together with components that normally accompany it in its natural state. Isolated material (e.g. viral particles and bacterial cells) may be in purified or partially purified form.

By “immune response” is meant the actions taken by a host to defend itself from pathogens or abnormalities. The immune response includes innate (natural) immune responses and adaptive (acquired) immune responses. Innate responses are antigen non-specific. Adaptive immune responses are antigen specific. An immune response in an organism provides protection to the organism against bacterial and viral infections when compared with an otherwise identical subject to which the composition or cells were not administered or to the human prior to such administration.

“Proliferation” is used herein to refer to the reproduction or multiplication of similar forms, especially of bacterial cells or the production of viral particles. That is, proliferation encompasses production of a greater number of bacterial cells or viral particles, and can be measured by, among other things, simply counting the numbers of bacterial cells, measuring incorporation of ³H-thymidine into bacterial cells, assessing the increased numbers of labeled bacterial cells and viral particles in in vitro cultures or specimens obtained ex vivo from subjects, detection of the presence and concentration of viral genomes, and the like.

A “protective immune response” against viral and bacterial infectious disease refers to an immune response exhibited by a subject (e.g., a human) that is protective against disease when the individual is subsequently exposed to and/or infected with wild-type viruses and bacteria. Typically, the protective immune response results in detectable levels of host engendered serum and secretory antibodies that are capable of neutralizing bacterial cells and viral particles of the same strain and/or subgroup (and possibly also of a different, non-vaccine strain and/or subgroup) in vitro and in vivo.

By “increases” is meant a positive alteration of at least 10%, 25%, 50%, 75%, 100%, 200%, 300%, 400%, 500%, 1000%, or more.

By “infection” is meant a bacterial colonization of the host. Infection of a host can occur by entry of a bacterial cells and viral particles through a break in barrier epithelial surfaces, such as injection by an infected tsetse fly, breaks in the skin, the eyes, or with the mucous membranes.

By “infectious disease” is meant a disease or condition in a subject caused by a pathogen that is capable of being transmitted or communicated to a non-infected subject. Non-limiting examples of infectious diseases include bacterial infections, viral infections, fungal infections, parasitic infections, and the like.

By “pathogen” is meant an infectious agent, such as a bacteria or virus, capable of causing infection, producing toxins, and/or causing disease in a host.

As used herein, “centanamycin” or “CM” refers to the compound:

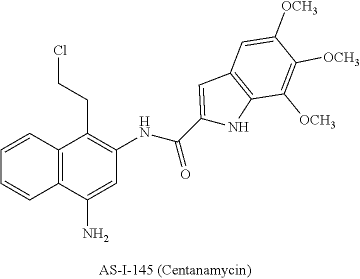 [SMILES: COc1cc2cc(C(=O)Nc3cc(N)c4ccccc4c3CCCl)[nH]c2c(OC)c1OC]

or a salt, solvate, geometric isomer, or stereoisomer thereof.

As used herein, “tafuramycin” refers to at least one of the compounds:

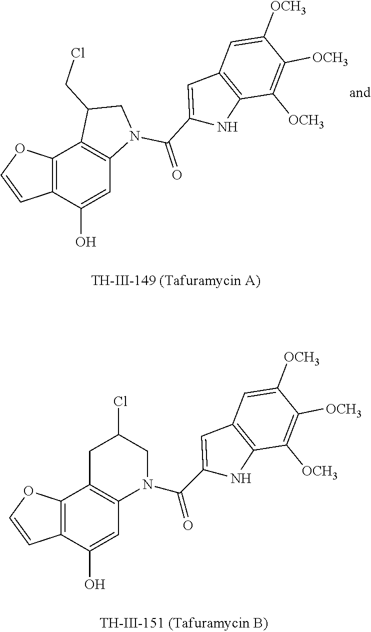 [SMILES: COc1cc2cc(C(=O)N3CC(CCl)c4c3cc(O)c3ccoc43)[nH]c2c(OC)c1OC ; COc1cc2cc(C(=O)N3CC(Cl)Cc4c3cc(O)c3ccoc43)[nH]c2c(OC)c1OC]

or salts, solvates, geometric isomers, or stereoisomers thereof.

By the term “modified” or “engineered” as used herein, is meant a changed state or structure of a molecule or cell of the disclosure. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

A “portion” of a polynucleotide means at least at least about twenty sequential nucleotide residues of the polynucleotide. It is understood that a portion of a polynucleotide may include every nucleotide residue of the polynucleotide.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

As used herein, “sample” or “biological sample” refers to anything, which may contain the cells or particles of interest (e.g., bacterial cells or viral particles) for which the screening method or treatment is desired. The sample may be a biological sample, such as a biological fluid or a biological tissue. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

A “subject” as used therein, may be a human or non-human mammal. Non-human mammals include, for example, primates, livestock and pets, such as bovine, porcine, ovine, canine, feline and murine mammals. Preferably, the subject is human.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e³ and e¹⁰⁰ indicating a closely related sequence

By “reference” is meant a standard or control condition.

A “reference sequence” is a defined sequence used as a basis for sequence comparison.

As used herein, the terms “treat,” treating,” “treatment,” and the like refer to reducing or improving an infectious disease or condition and/or one or more symptoms associated therewith. It will be appreciated that, although not precluded, treating an infectious disease or condition and/or one or more symptoms associated therewith does not require that the disorder, condition, disease or symptoms associated therewith be completely ameliorated or eliminated.

By the term “vaccine” as used herein, is meant a composition, a protein, or a nucleic acid of the invention, which serves to protect an animal against an infectious disease and/or to treat an animal having an infectious disease compared with an otherwise identical animal to which the vaccine is not administered or compared with the animal prior to the administration of the vaccine.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., retroviruses, adenoviruses, and adeno-associated viruses) that incorporate the recombinant polynucleotide.

By “virulence” is meant a degree of pathogenicity of a given pathogen or the ability of an organism to cause disease in another organism. Virulence refers to an ability to invade a host organism, cause disease, evade an immune response, and produce toxins.

By “virulent” or “pathogenic” is meant a capability of a bacterium to cause a severe disease.

By “non-pathogenic” is meant an inability to cause disease.

By “wildtype” is meant a non-mutated version of a gene, allele, genotype, polypeptide, or phenotype, or a fragment of any of these. It may occur in nature or be produced recombinantly.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Description

In one aspect, the invention of the disclosure is based on the discovery that bacterial cells and DNA virus particles can be treated with centanamycin, tafuramycin, duocarmycin, or related compounds, such that the cells or virus particles remain viable but are replication-deficient. Immunogenic compositions comprising these treated cells and particles can then be used as vaccines to immunize subjects against the bacterial cells or viruses or combinations thereof.

Accordingly, in one aspect, the invention includes a method of producing an immunogenic composition against a pathogen, wherein the pathogen is a virus or bacterium.

In certain embodiments, the method comprises providing isolated or purified particles and/or cells of a pathogen, or infected cells comprising the pathogen. In certain embodiments, the method comprises contacting the pathogenic particles and/or cells with centanamycin, or a salt, solvate, analog, or derivative thereof, thereby rendering the pathogenic particles or cells attenuated and replication-defective. In certain embodiments, the method comprises isolating the replication-defective pathogenic particles or cells, thereby producing the immunogenic composition.

### DNA Alkylating Agents

Centanamycin, also known as ML-970, AS-I-145, D716970, and NSC 716970, is a indolecarboxamide that irreversibly alkylates the DNA by binding covalently to the adenine-N3 in the A-T rich sequence motif (A/T)AAA in the minor groove of the DNA. The alkylation of the DNA molecule thus results in the inhibition of DNA replication. Centanamycin has proven to be a potent anti-malarial drug for developing a whole parasite vaccine. Many studies have proven centanamycin efficacy in generating whole parasite vaccine for malaria and has been patented for malaria vaccine (U.S. Pat. No. 9,539,316). Moreover, centanamycin has proven to be a potent anti-cancer alkaloid which covalently alkylates A-T sequence in the minor groove of the DNA. However, due to the toxicity of centanamycin and its related compounds, its use in vivo is difficult, limiting its use as an anti-cancer drug.

However, this offers an effective strategy for the use of centanamycin and its analogs and derivatives in attenuating bacterial cells and viral particles under in vitro conditions to generate live replication-defective viruses for the development of effective vaccines. The live attenuated viruses which can infect the cells but are not able to replicate further due to their damaged DNA are an ideal vaccine for generating an effective immune response. Likewise, whole, attenuated bacterial cells can be used in a similar matter. These live attenuated viruses and bacteria have all proteins that can be recognized by the immune system presented in an immune context sufficient to generate enough memory cells to prevent subsequent infections with the viruses and bacteria. These attenuated viruses and bacterial cells cannot escape from the body immune system due to their damaged DNA and thus represent effective and safe strategies for treatment. In some embodiments of the current invention, the centanamycin treatment is used on DNA viruses such as human cytomegalovirus, varicella zoster virus, and human simplex virus to generate live-attenuated vaccine compositions.

In some embodiments, the current invention includes the use of compounds that are related to or derivatives of centanamycin and can include, but are not limited to TfA (tafuramycin A) (C₂₃H₂₁ClN₂O₆ and 456.88 g/mol), TfB (tafuramycin B) (C₂₃H₂₁ClN₂O₆ and 456.88 g/mol), and HxTfA (duocarmycin analog) (C₂₆H₂₀ClN₃O₄ and 473.91 g/mol) among others. Tafuramycin A is an AT-containing DNA binding and alkylating agent based on duocarmycins that comprises a stereocenter.

### Compounds

The present disclosure provides certain compounds, or a salt, solvate, geometric isomer, or stereoisomer thereof.

In certain embodiments, the compound is at least one of:

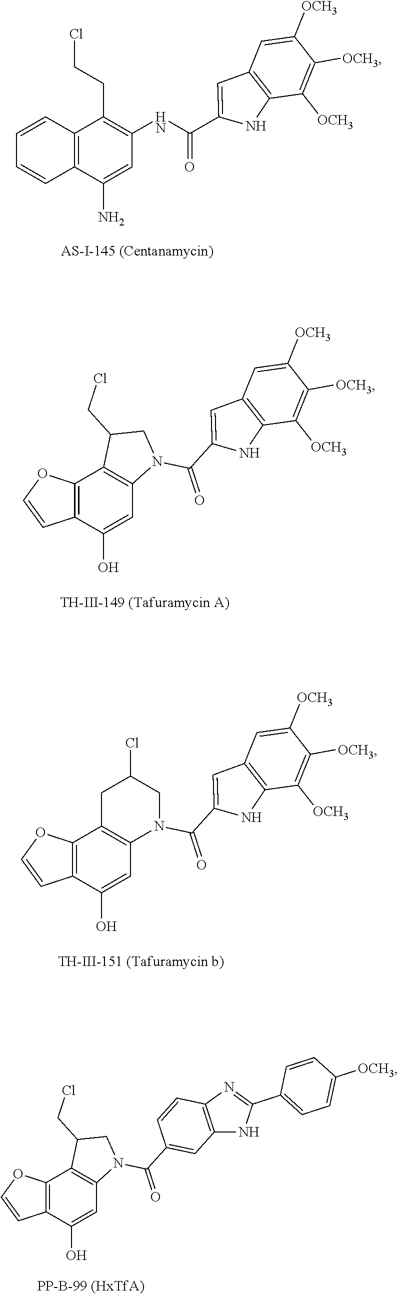 [SMILES: COc1cc2cc(C(=O)Nc3cc(N)c4ccccc4c3CCCl)[nH]c2c(OC)c1OC ; COc1cc2cc(C(=O)N3CC(CCl)c4c3cc(O)c3ccoc43)[nH]c2c(OC)c1OC ; COc1cc2cc(C(=O)N3CC(Cl)Cc4c3cc(O)c3ccoc43)[nH]c2c(OC)c1OC ; COc1ccc(-c2nc3ccc(C(=O)N4CC(CCl)c5c4cc(O)c4ccoc54)cc3[nH]2)cc1]

or a salt, solvate, geometric isomer, or stereoisomer thereof.

In certain embodiments, the Centanamycin, Tafuramycin A, Tafuramycin B, or an analog or derivative thereof is a compound of Formula (I):

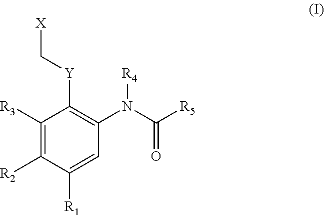

wherein,

R₁ is NHR or OR, where R is selected from H, benzyl, benzyloxycarbonyl, 4-nitrobenzyloxycarbonyl and N′-methylpiperazinyl-N-carbonyl;

R₂ is selected from H and C₁₋₆ alkyl;

R₃ is selected from H and C₁₋₆ alkyl;

or R₂ and R₃ form a fused ring selected from the group consisting of benzene, pyrrole, pyridine, furan, and 5-methylfuran, which fused ring may be optionally substituted with C₁₋₆ alkyl, CF₃, or C₁₋₆ alkyloxycarbonyl;

Y is —CH— to form a five-membered ring with R₄;

X is an electrophilic leaving group;

R₄ is a —CH₂— group bonded to Y to form a five-membered ring; and

R₅ is selected from the group consisting of:

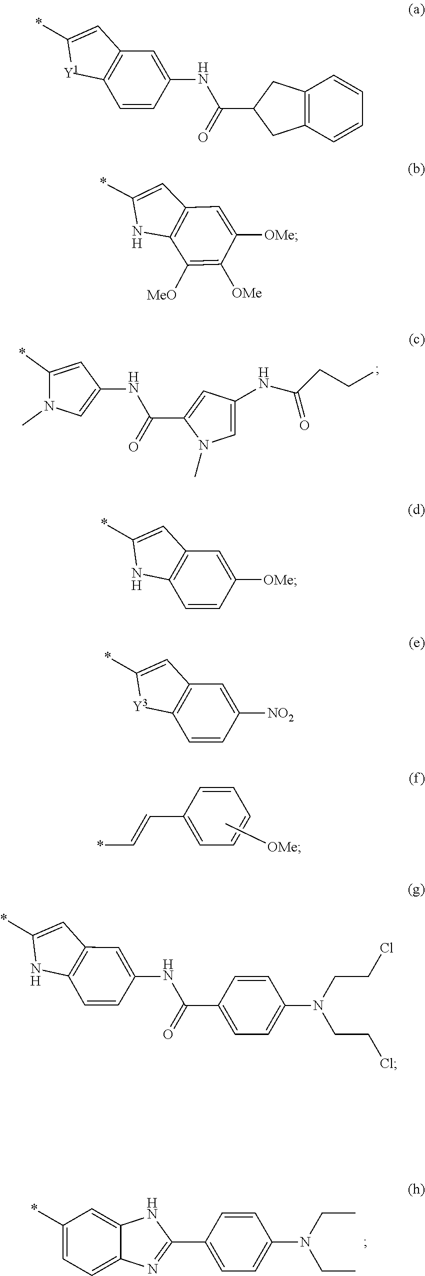

wherein Y¹ and Y² are independently selected from O and NH and wherein Y³ is independently selected from O and NH.

In some embodiments, the Centanamycin, Tafuramycin A, Tafuramycin B or an analog or derivative thereof is a compound of Formula (II):

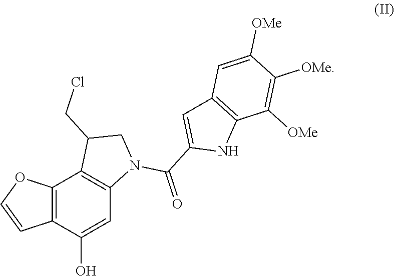 [SMILES: COc1cc2cc(C(=O)N3CC(CCl)c4c3cc(O)c3ccoc43)[nH]c2c(OC)c1OC]

In certain embodiments, the Centanamycin, Tafuramycin A, Tafuramycin B or an analog or derivative thereof is a compound of Formula (III):

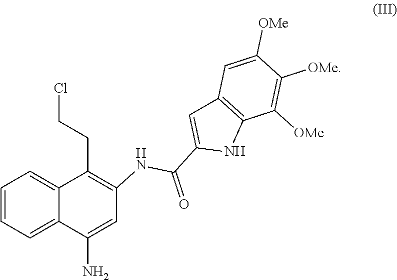 [SMILES: COc1cc2cc(C(=O)Nc3cc(N)c4ccccc4c3CCCl)[nH]c2c(OC)c1OC]

In certain embodiments, the Centanamycin, Tafuramycin A, Tafuramycin B or an analog or derivative thereof is a compound of Formula (IV):

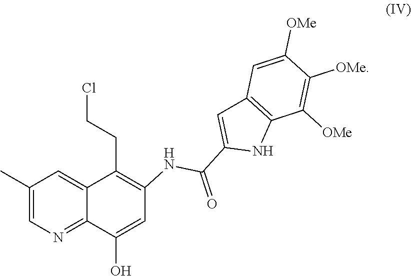 [SMILES: COc1cc2cc(C(=O)Nc3cc(O)c4ncc(C)cc4c3CCCl)[nH]c2c(OC)c1OC]

Typical, although non-limiting concentrations of Centanamycin, Tafuramycin A, Tafuramycin B or analogs or derivatives for the attenuation of bacterial cells and viral particles are in the range 0.000001 to 1,000 μM. Preferably, the concentration is less than 5 μM, more preferably less than 2 μM or in the range about 0.1-1 μM or about 0.2 μM. Treatment duration may be in the range 1 minute to 12 hours, preferably 10 minutes to 4 hours or more preferably about 0.5, 1, 1.5 or 2 hours.

The compounds of the invention may possess one or more stereocenters, and each stereocenter may exist independently in either the (R) or (S) configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or mixtures thereof, or in the case where two or more chiral center are present, all diastereomers or mixtures thereof.

In certain embodiments, the compounds of the invention exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C , ¹³C , ¹⁴C, ³⁶Cl, ¹⁸F, ¹²³I, ¹²⁵I, ¹³N, ¹⁵N , ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed invention. The compounds of the invention may contain any of the substituents, or combinations of substituents, provided herein.

### Salts

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Compositions

In certain aspects, the current invention provides an immunogenic composition comprising a pharmaceutically acceptable carrier or diluent and an effective amount of isolated or purified particles and/or cells from a pathogen, wherein the isolated or purified pathogenic particles and/or cells are attenuated and replication-defective. In this way, the immunogenic composition is capable of inducing an effective immune response against the purified particles and/or cells in a subject to which the immunogenic composition is administered. An effective immune response is defined as an immune response that results in significant immune memory sufficient to give protection against subsequent exposure to the pathogen and closely related pathogens.

In certain embodiments, the isolated or purified pathogenic particles and/or cells are rendered attenuated and replication-deficient due to treatment with a compound selected from centanamycin or tafuramycin A, or a salt, solvate, analog, or derivative thereof. In this way, the immunogenic composition maintains the purified pathogenic particles and/or cells largely intact though unable to be actively infectious. The intact nature of the immunogenic composition aids in the development of effective immunity against antigens, especially surface antigens, which are readily accessible by the subject's immune system.

In certain embodiments of the current invention, the pathogen comprising the immunogenic composition can be a virus, a bacterium, or any combination of the two. In certain embodiments, the pathogen is a virus, preferably a DNA virus. DNA viruses are viruses whose genomes are comprised of either single- or double-stranded DNA which is replicated by either a viral or host DNA-dependent DNA polymerase or can be replicated through an RNA intermediate via reverse transcription. Immunogenic compositions of the invention can be comprised of one or more pathogenic DNA viruses. Examples of DNA viruses that can comprise the immunogenic compositions of the invention include but are not limited to viruses of the adenoviridae, papovaviridae, parvoviridaem, herpesviridae, poxviridae, anelloviridae, papillomaviridae, and pleolipoviridae families. In certain embodiments, the immunogenic compositions of the current invention comprise any combination of specific DNA viruses, including but not limited to cytomegalovirus including human and mouse cytomegaloviruses, Herpes simplex viruses including HSV types 1 and 2, Human alphaherpesvirus 3 (varicella-zoster virus), Epstein-Barr virus, varicella virus, Human papilloma virus, hepatitis B virus, human herpes viruses, among others.

In certain embodiments of the current invention, the pathogen comprising the immunogenic composition can be bacterium or mixture of bacteria. Bacterial cells possess DNA genomes, which make them suitable for treatment with the DNA alkylating compounds of the current disclosure to render them attenuated and replication-deficient. It is contemplated that the immunogenic compositions of the current invention can comprise bacterial cells of any number of genuses including, but not limited to Escherichia including Escherichia coli, Mycobacterium, Streptococcus, Pseudomonas, Staphylococcus, Chlamydia, Neisseria, Borrelia including Borrelia burgdorferi, Brucella, Listeria, Legionella, Shigella, Campylobacter, Salmonella, and any combination thereof.

In certain embodiments, it is contemplated that the immunogenic composition of the current invention can comprise one or more bacteria and/or one or more viruses.

### Methods of Use

In some aspects, the invention includes centanamycin- or tafuramycin A- or tafuramycin B-treated bacterial and/or viral particles. These treated cells and/or particles may be used to prepare the immunogenic composition as an isolated, purified or partially purified preparation, which are then used to prepare the immunogenic composition. Immunogenic compositions may be comprised of a mixture of bacterial cells and viral particles from one or more species of bacteria and viruses.

The methods and compositions provided herein can be used to stimulate or generate an immune response in a subject against a subsequent infection caused by the same types of bacterial cells and/or viral particles used in the creation of the immunogenic compositions of the invention. Such immune responses can be comprised of antibodies and T cells specific for epitopes derived from the bacterial cells and/or viral particles of the immunogenic compositions.

The methods include administering an immunologically effective amount of an immunogenic composition provided herein to a subject in a physiologically acceptable carrier. Typically, the carrier or excipient for vaccines provided herein is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, and the like.

The methods also include administering an adjuvant, such as an oil-in-water emulsion, a saponin, a cholesterol, a phospholipid, a CpG, a polysaccharide, variants thereof, and a combination thereof, with the composition of the invention. Optionally, a formulation for prophylactic administration also contains one or more adjuvants for enhancing the immune response to the bacterial or viral antigens. Suitable adjuvants include: complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, bacille Calmette-Guerin (BCG), Corynebacterium parvum, and the synthetic adjuvants QS-21 and MF59.

Pharmaceutical formulations that are useful in the methods of the invention may be suitably developed for inhalational, oral, parenteral, pulmonary, intranasal, intravenous or another route of administration. Other contemplated formulations include projected nanoparticles, liposomal preparations, and immunologically-based formulations. The route(s) of administration will be readily apparent to the skilled artisan and will depend upon any number of factors including the type and severity of the disease being treated, the type and age of the veterinary or human patient being treated, and the like.

The pharmaceutical formulations described herein may be prepared by any method known or hereafter developed in the art of pharmacology. In general, such preparatory methods include the step of bringing the cells into association with a carrier or one or more other accessory ingredients, and then, if necessary or desirable, shaping or packaging the product into a desired single- or multi-dose unit.

In one embodiment, the cells and/or particles of the invention are formulated using one or more pharmaceutically acceptable excipients or carriers. In one embodiment, the pharmaceutical formulations of the cells of the invention include a therapeutically effective amount of the cells of the invention and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

### Administration/Dosing

In the clinical settings, delivery systems for the compositions described herein can be introduced into a subject by any of a number of methods, each of which is familiar in the art. For instance, a pharmaceutical formulation of the composition can be administered by inhalation or systemically, e.g. by intravenous injection.

The regimen of administration may affect what constitutes an effective amount. The therapeutic formulations may be administered to the subject either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the composition of the present invention to a subject, preferably a mammal, more preferably a human, may be carried out using known procedures, at dosages and for periods of time effective to treat a disease or condition in the subject. An effective amount of the composition necessary to achieve a therapeutic effect may vary according to factors such as the extent of implantation; the time of administration; the duration of administration; other drugs, compounds or materials used in combination with the composition; the state of the disease or disorder; age, sex, weight, condition, general health and prior medical history of the subject being treated; and like factors well-known in the medical arts. Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of the composition without undue experimentation.

Actual dosage levels of the cells in the pharmaceutical formulations of this invention may be varied so as to obtain an amount of the composition that are effective to achieve the desired therapeutic response for a particular subject, composition, and mode of administration, without being toxic to the subject.

### Routes of Administration

Routes of administration of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable formulation of the composition sand dosages include, for example, dispersions, suspensions, solutions, beads, pellets, magmas, creams, pastes, plasters, lotions, discs, suppositories, liquid sprays for nasal or oral administration, aerosolized formulations for inhalation, compositions and formulations for intravesical administration and the like.

It should be understood that the formulations and compositions that would be useful in the present invention are not limited to the particular formulations set forth in the examples. The following examples are put forth so as to provide those of ordinary skill in the art with a complete disclosure and description of how to make and use the cells, differentiation methods, engineered tissues, and therapeutic methods of the invention, and are not intended to limit the scope of what the inventors regard as their invention.

### Kits

The invention provides kits for the treatment or prevention of a bacterial or viral infection. In one embodiment, the kit includes a therapeutic or prophylactic composition containing an effective amount of an immunogenic composition (e.g., centanamycin-treated bacterial cells or viral particles or a mixture thereof) in unit dosage form. In some embodiments, the kit comprises a device (e.g., nebulizer, metered-dose inhaler) for immunogenic composition dispersal or a sterile container which contains a therapeutic or prophylactic immunogenic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired an immunogenic composition of the invention is provided together with instructions for administering the immunogenic composition to a subject having or at risk of contracting or developing a bacterial or viral infection. The instructions will generally include information about the use of the composition for the treatment or prevention of a bacterial or viral infection. In other embodiments, the instructions include at least one of the following: description of the therapeutic/prophylactic agent; dosage schedule and administration for treatment or prevention of bacterial or viral infection or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out embodiments of the present invention, and are not to be construed as limiting in any way.

The materials and methods used in the experimental examples of the present invention are now described.

Viruses, cells lines, and animals: The viruses used in this study were ToledoLuc, VZV-GFP-Luc, MCMV Luciferase (MCMVLuc, Smith strain), HSV1 KOS. The mammalian cell lines used in this work were mouse 3T3 fibroblast cells, human retinal pigment epithelial ARPE-19 cells (ATCC CRL-2302), and human lung fibroblast MRC-5 cells (ATCC, #CCL-171). The ARPE-19 and MRC-5 cells were grown in Dulbecco's modified Eagle medium (DMEM) containing 50 U/ml penicillin, and 50 μg/ml streptomycin (Invitrogen), and 10% fetal bovine serum (FBS, Hyclone). The 3T3 cells were grown in minimum essential medium (Gibco) with 50 U/ml penicillin, and 50 μg/ml streptomycin (Invitrogen), and 10% fetal bovine serum (FBS, Hyclone). 6-8 weeks old BALB/C mice were used in this study.

Centanamycin stock: A 3.3 mM stock of CM was prepared in PET solution (polyethylene glycol 400, absolute ethanol, and Tween-80 in 6:3:1 ratio)/glucose solution. The working stock of CM for virus treatment was obtained by diluting the 3.3 mM stock in PET/glucose solution (6:3:1).

ToledoLuc treatment with CM: The human cytomegalovirus (HCMV) strain ToledoLuc, which contains a luciferase expression cassette, was used in this study and well-known to those of skill in the art. Cells of the MRC-5 human diploid fibroblast line were seeded into 12-well plates. The Toledo-Luc virus was treated with 10 μM, 1 μM, 0.1 μM, 0.01 μM, and 0.001 μM of CM for 2 hours at room temperature. After 2 hours of treatment with CM, ToledoLuc viruses were washed 3 times with cDMEM and added to MRC-5 cells at an MOI of 0.05 in triplicate wells. The control includes the ToledoLuc treated with the PET vehicle in a similar way. The growth kinetics of ToledoLuc was determined by bioluminescence assay. The luciferase activity of ToledoLuc virus was measured every 24 hours using IVIS-50.

Fluorescence microscopy: The AD169-GFP viruses were incubated with 100 μM, 10 μM, 1.0 μM, and 0.1 μM of centanamycin as described above. After treatment, the AD169-GFP viruses were washed 3 times with the cDMEM and added to the ARPE-19 cells at an MOI of 0.05. The virus growth was analyzed every day by fluorescence microscopy. In a control experiment, the viruses were only treated with the PET vehicle.

Human Simplex Virus (HSV) treatment with CM: The ARPE-19 (ATCC CRL-2302) cells were grown in 12 well plate in DMEM with 10% FBS and 1% P/S. HSV-1 were treated with different concentration of centanamycin (100 μM, 10 μM, 1μM, 0.1 μM, and 0.01 μM) for two hours at room temperature. The CM treated HSV were washed 3 times with cDMEM after treatment to remove CM and added to the ARPE-19 cells in triplicate at an MOI of 0.2. The HSV viruses were collected at 6, 12, 18, 24, 30, 36, and 42 hours of post-infection. The virus titer was determined by plaque assays using ARPE-19 cells.

Cell-free Varicella-zoster virus (VZV) treatment with the CM: VZV is a cell-associated virus. The efficacy of CM on VZV was checked using growth curve analysis. VZV-GFP-Luc was used to check the effect of CM on the virus. The ARPE-19 cells were seeded in 6-well plate and maintained at 37° C. and 5% CO₂. The cell-free virus was treated with different concentration of CM (centanamycin (100 μM, 10 μM, and 0.1 μM) for two hours at RT and washed with three times with cDMEM prior to adding to the cells. The virus load in the infected cells was determined by adding luciferin substrate and measuring relative luciferase units every 24 hours of post-infection. The control included the cells infected with the virus without CM treatment. Viability of infected cells was quantified using the Cell Counting Kit-8 (Sigma# 96992) according to the manufacturers protocol.

Analysis of CM and viral gDNA: To see any effect of CM on the HCMV gDNA, the gDNA was analyzed by agarose gel electrophoresis, restriction digestion, and thermal incubation at 50° C. The HCMV was grown in ARPE-19 cells and virus particles were purified through sucrose gradient using ultracentrifugation. HCMV gDNA was extracted and concentration was determined by OD at 260/280. Approximately, 10 μg of HCMV gDNA was incubated with 10 μM CM at RT for 1 hour and 10 μg of HCMV gDNA was treated with vehicle control without CM. The treated gDNA was analyzed by the agarose gel electrophoresis to see any effect of CM on the HCMV gDNA. In a separate experiment, the and 100 μM treated HCMV gDNA was incubated at 50° C. for 30 minutes to check the alkylation of DNA is thermolabile or not. Untreated HCMV gDNA was also treated similarly. In another, 3 μg of CM treated and untreated HCMV gDNA was digested with EcoR1 overnight at 37° C. and analyzed by 0.6% agarose gel electrophoresis.

Identification of CM nucleotide adduct in viral DNA by LC-MS: 5 μg (50 μl) of HCMV gDNA was Incubated with 10 μM of CM for two hours at room temperature followed by 30 minutes at 50° C. treatment. The CM adduct of nucleotides was extracted using ethanol acetate extraction 4-5 times. The extracted CM-nucleotide adduct was allowed to vaporize in the fume hood and a residue was dissolved In the HPLC grade acetonitrile and filter through a 0.2μ filter. The LC-MS was performed to Identify the CM-nucleotide adduct using

In vivo experiments: To check the efficacy of CM in the animal model, mouse cytomegalovirus expressing the luciferase gene (MCMVLuc) was used. The in vitro results suggested that 0.1 μM CM was efficient in generating attenuated viruses. The 25×10⁶ PFU/ml of MCMVLuc viruses were treated with 10 μM, 1.0 μM, and 0.1 μM of CM for two hours at room temperature. The MCMVLuc viruses were washed with RPMI to remove CM and suspended in 1 ml of incomplete RPMI. Thereafter, 200 μl of the CM treated MCMVLuc was injected into five mice per group. The luciferase activity was measured every other day using bioluminescence assay by an in vivo imaging system (IVIS). Briefly, the mice were anesthetized using isoflurane and injected with the luciferin substrate. The injected mice were incubated at RT for five minutes and luciferase activity was determined using IVIS. In control experiments, the MCMVLuc viruses were treated with PET only and followed in a similar way as described above.

Immunization with chemically live-attenuated replication-defective virus: For each immunization, 6×10⁶ PFU of MCMV were treated with 0.1 μM CM for two hours at RT followed by two washing with DMEM to remove unbound CM. Five BALB/C mice per group were immunized with the attenuated MCMV (1×10⁶ PFU per mouse) along with alum adjuvant. The second and third immunization was performed with 5×10⁵ PFU/per mouse. Subcutaneous immunization was performed on day 1, day 15, and day 30. Pre-bleed was performed before immunization and serum were stored at −80° C. until use. After 15 days of last immunization, 100 μl of blood was collected and allowed to clot at RT. Blood samples were centrifuged and serum was collected for storage at −80° C. until use. The mice immunized with DMEM/PBS/alum adjuvant only were used as a control and treated in a similar manner.

Virus-specific antibodies response in immunized mice: The serum collected after 15 days of last immunization of the mice were analyzed by ELISA to see if there were MCMV specific antibodies. The serum was diluted in 1% BSA in a ratio of 1:100, 1:500, 1:1000, 1:2000, 1:4000, 1:8000, 1:16000, and 1:32000. The MCMV virus particles (50 μl/per well) were coated in carbonate buffer into the 96 wells plate overnight at 4° C. The uncoated viruses were washed with 0.1% PBST and blocked 3% BSA in PBST at 37° C. for 2 h. The diluted serum was added in triplicates to analyze MCMV specific antibody response. The plate was incubated at RT for 2 hours and washed three times with 1XPBST (0.1% Tween 20). Secondary antibody diluted in 3% BSA (1:1000) was added to each well and incubated for 1 hour at RT. Thereafter, the plate was washed to remove the unbound secondary antibodies. The alkaline phosphatase was added to each well and plates were read at 410 nm in an ELISA reader.

Virus neutralization assay: NIH 3T3 cells were seeded into 24-well plates. The serum from immunized and control mice was used for virus neutralization assay. Serially diluted serum (2-fold) was added to the 10,000 PFU of MCMVLuc and incubated at RT for one hour. After incubation, MCMVLuc virus was added to the 3T3 cells and viral load was determined by RLU (relative luminescence units) in the IVIS machine. The neutralization titer 50 (NT50) was determined by calculating the antibody titer that required to neutralize 50% of the virus load.

Mice challenge assay: Fifteen days following the third immunization, mice in each group were challenged with 1×10⁴ PFU of MCMV intraperitoneally. After a further 10 days following virus administration, salivary glands were isolated from challenged mice and tissue homogenate was prepared to determine the viral titer using a plaque assay. To serve as a control, unimmunized mice challenged were with 1×10⁴ PFU of MCMV an sacrificed on day 10, followed by isolation of the salivary glands. For the plaque assay, tissue lysate was diluted serially and added to cultures of 3T3 cells seeded into six-well plates. The virus titer was determined for tissue homogenate prepared from each mouse.

CM treatment of E. coli: Overnight cultures of E. coli culture were diluted 200 times in LB media to make final volume of 1 ml and treated with different concentrations (100 μM, 10 μM, 1 μM, and 0.1 μM) of centanamycin for three hours at room temperature. The treated E. coli cells were then centrifuged at 10,000 rpm for 1 min, supernatant was removed and the pellet was resuspended in 100 μl of LB and plated on the LB agar plate without any antibiotic. The plates were then incubated at 37° C. for 16 hours. To determine the effect of centanamycin, the number of colonies were counted on each plate.

E. coli growth assay: E. coli cultures were established overnight, after which 10 μl if each culture was added to 100 μl of LB broth and treated with different concentration of CM (100 μM, 10 μM, 1 μM, and 0.1 μM) for two hours at RT. Replicate samples were performed in triplicate. The treated bacterial cells were then added to 96 well, round-bottom plates, which were then incubated at 37° C. in a shaking incubator. The bacterial OD was measured in Glomax Explorer (Promega) every hour to determine the bacterial growth.

The experimental examples are now described.

### EXAMPLE 1

### Generation of CM-Attenuated Live Cytomegalovirus

Human cytomegalovirus (HCMV) is a ubiquitous betaherpesvirus that causes asymptomatic infection in healthy individuals. However, HCMV infection can have severe consequences in immunocompromised and organ transplant patients. Moreover, congenital HCMV infection causes fetal malformation and is the leading cause of the birth defect in new borne. Moreover, CMV has been associated with many forms of cancers such as breast cancer, glioblastoma, colorectal cancer, among others.

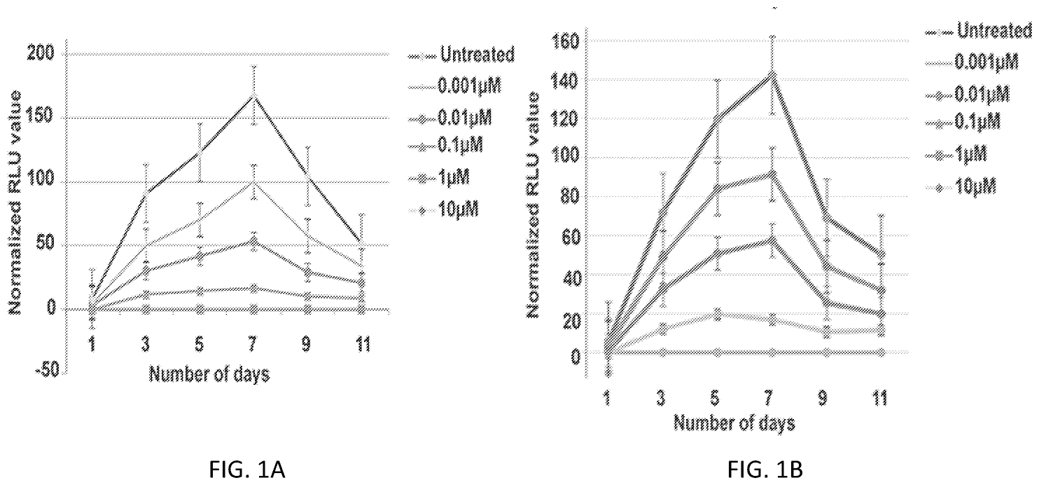

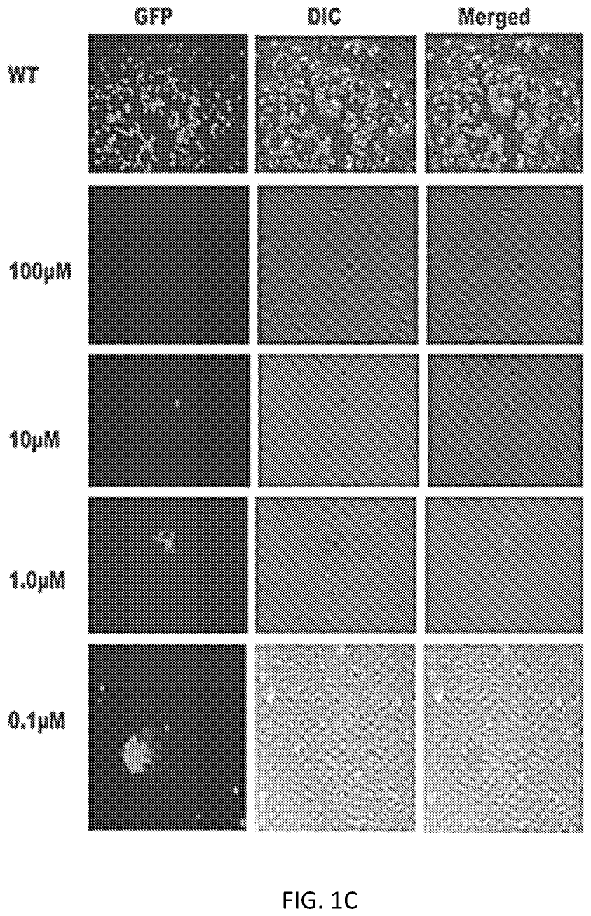

In the present example, the effect of CM treatment on the HCMV was first examined using the modified ToledoLuc strain of HCMV. ToledoLuc was treated with different concentrations of CM for two hours at room temperature, washed to remove unbound CM and then allowed to grow in the MRCS cells. The results demonstrated that Toledo-luc virus does not replicate when treated with the 10 μM and 1 μM CM (FIG. 1A). In 100 μM and 10 μM of CM, Toledo-luc neither replicate nor infects the cells indicating a complete death of the virus (FIG. 1A). This result suggested that 10 μM CM was killing the virus in 2 hours incubation at RT, possibly from damage of the viral DNA. Luciferase activity was not detected in the infected cells after 24 hours of infection until 9 days post-infection (FIGS. 1A-1B). ToledoLuc virus treated with 1 μM CM for 2 hours at RT did not grow and was replication-defective when added to MRC5 cells, and did not result in any luciferase activity in the infected cells 9 days post-infection (FIGS. 1A-1B). These results demonstrated that 100 μM, 10 μM and 1 μM CM are most potent in preventing CMV infection and replication. ToledoLuc treated with the 0.1 μM CM for 2 hours at RT, allow the virus to infect the cells but did not replicate as demonstrated by the luciferase activity in the infected cells. These results indicated that 0.1 μM CM treatment of ToledoLuc allow the virus to infect the cells and virus got mutated to an extent that does not allow the virus to replicate. This is a novel opportunity for the design of an attenuated vaccine where a chemical treatment allows the virus to infect cells but does not replicate. Furthermore, these results confirmed that treatment with 0.01 μM-0.001 μM allows the virus to grow and replicate in the infected cells suggesting that Toledo-Luc virus can escape from these concentrations of CM.

To understand the pathology of CM treated virus, a GFP-expressing human cytomegalovirus, AD169-GFP, was used with microscopic imaging to visually assess the ability of treated viruses to infect cell. Results from these studies demonstrated that 100 μM CM treated viruses were not detected in the infected cells when observed by fluorescence microscopy (FIG. 1C). This indicated that the virus was not able to infect the cells, suggesting a complete inactivation of the treated virus. AD169-GFP treated with 10 μM CM were not able to infect the cells as suggested by a very few GFP positive cells (FIG. 1C). The viruses which entered in cells did not grow or replicate. At 1 μM, treated virus was detected in the infected cells but did not replicate as suggested by the GFP signal was confined to one cell as compared to the untreated virus which replicated efficiently. This also suggested that 1 μM CM treatment caused the virus to mutate to such a magnitude that allows the virus to infect the cells but did not allow the virus to replicate due to damaged DNA (FIG. 1C). The results obtained from Toledo-luc and AD169-GFP virus treatment strongly suggest that CM is an effective drug to generate chemically attenuated CMV under in vitro conditions.

### EXAMPLE 2

### HSV-2-GFP Treatment with CM

To determine if CM can attenuate other DNA viruses, a series of studies was then conducted in order to evaluate effects of CM treatment on the human simplex virus-2 expressing GFP (HSV-2 GFP). At present, there is no vaccine for HSV-1 and HSV-2 infections. HSV-1 and HSV-2 are human pathogens causing mucosal infections and establish latent infection in sensory neurons. These viruses reactivate to cause disease and manifest severe complications. Immunocompromised and AIDS patients are especially at risk of life-threatening reactivation of HSVs. The HSV infections and genital herpes pandemic severity in HIV infected persons require an effective vaccine for HSV treatment.

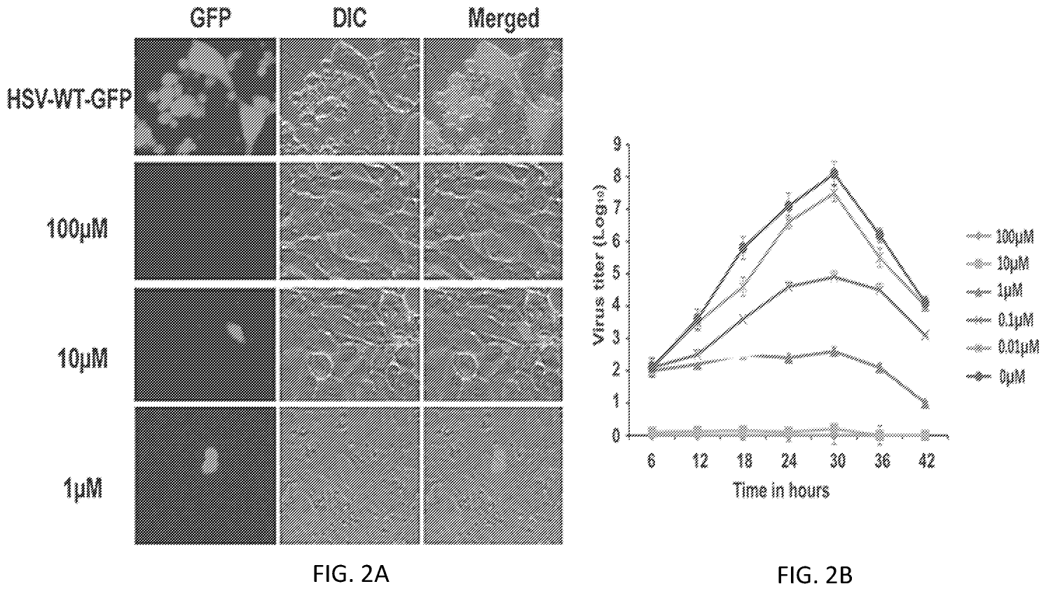

In this study, the HSV-2 GFP was treated with the different concentrations of CM ranging from 100 μM to 0.01 μM (10-fold serial dilution at each concentration) and determined the growth curve using plaque assay (FIG. 2B). The results obtained from the growth curve analysis of HSV-2-GFP treated with CM confirmed that 100 μM CM concentration completely inhibits the virus infection in ARPE-19 cells (FIG. 2A). A 10 μM CM treatment of HSV-2-GFP leads to the virus attenuation and the virus was not able to spread to adjacent cells (FIG. 2A). HSV-2-GFP treatment with 1.0 μM caused slower growth as compared to untreated. The growth curve analysis shows a dose-dependent growth of the HSV-2-GFP in response to the CM (FIG. 2B). Consistent with the microscopic results, 100 μM treated HSV-2-GFP was not detected in the infected cells indicating complete inactivation of the virus (FIG. 2B). The virus was able to infect the cells but not replicate when treated with 10 μM CM suggesting chemical attenuation of the virus (FIG. 2B). The virus treatment with 1 μM CM lead to viral infection in host cells and extremely slowly spread to neighbor cells (FIG. 2A). These results suggest that CM can attenuate HSV-2, and possibly other types of DNA viruses as well.

### EXAMPLE 3

### CM Inhibits the Growth of VZV in In Vitro Conditions

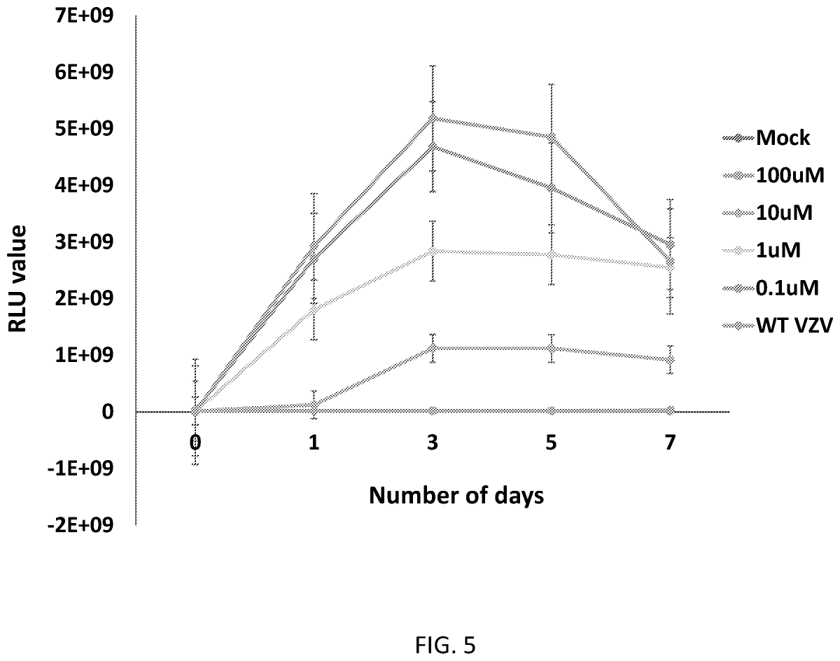

Subsequent studies then evaluated the effect of CM treatment on the replication and growth of human alphaherpesvirus-3, also known as varicella zoster virus (VZV), under in vitro conditions in human epithelial ARPE-19 cells. The VZV is a highly cell associated virus and it is transmitted by cell-cell spread. The cell-free, luciferase-expressing VZV-Luc was treated with different concentration of CM (100 μM, 10 μM, 1.0 μM, and 0.1 μM) for two hours at RT. After two hours, the virus was added to the ARPE-19 cells. Results demonstrated that the 100 μM CM treatment can completely inhibit the growth as well as replication of VZV-Luc (FIG. 5). A 10.0 μM CM treatment allowed the virus to infect the cells but not to spread to the adjacent cells. This suggested that 10.0 μM CM treatment generates an attenuated VZV-Luc which can infect the cells but not spread and forms the basis of attenuated and replication defective viral vaccine. The VZV-Luc treatment with 1.0 μM and 0.1 μM CM treatment allows the VZV-Luc to infect as well as spread to the adjacent cells but with reduced growth. This suggest that 1 μM and 0.1 μM CM concentrations are not able to attenuate the VZV completely. As compared to the CMV which can be attenuated with 1 μM CM, VZV is attenuated with 10 μM CM and this is because VZV is a cell associated virus and CM needs to cross the cell membrane to bind the virus and completely attenuates the VZV.

### EXAMPLE 4

### CM Attenuates Viral Particles through its Effects on DNA Alkylation

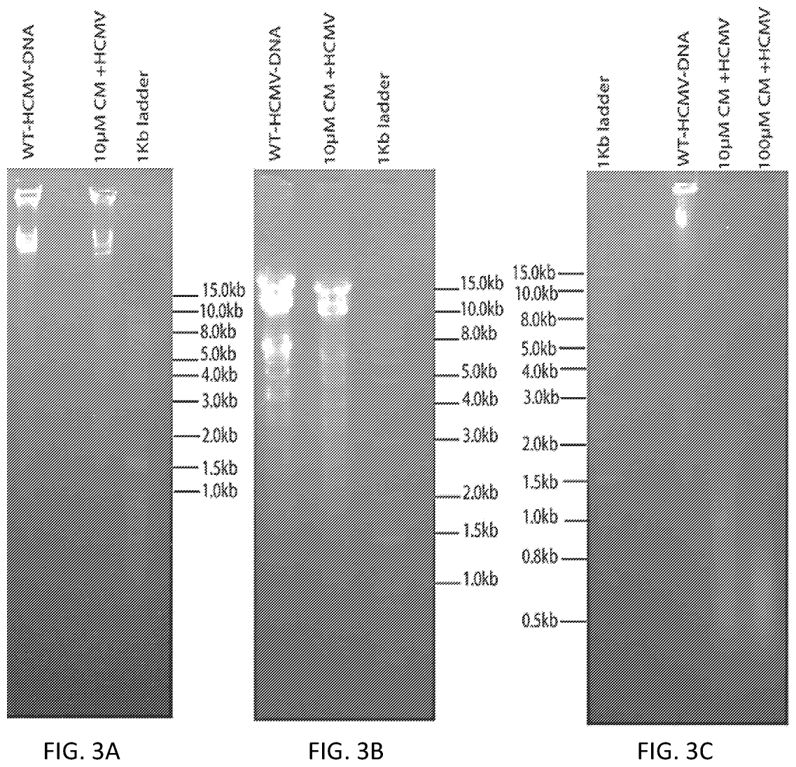

To understand the mechanism behind CM mediated attenuation of the virus, CM-treated viral gDNA was analyzed using agarose gel electrophoresis, restriction digestion, and heat-labile fragmentation assays. The AD169 viral gDNA was treated with 1 μM CM for two hours at RT and then separated with a 0.6% agarose gel. Untreated AD169 viral gDNA was used as a control. The results demonstrated that there were no big differences in the viral gDNA bands, mobility, and gel pattern of treated and untreated viral gDNA but there was a difference in the intensity of each band in treated DNA samples as compared to the control (FIG. 3A). To further understand the effect of CM on the viruses, viral gDNA was isolated and treated with CM for two hours at RT followed by digestion with the restriction enzymes EcoR1. The digestion pattern showed that CM treated DNA has no major missing bands due to the alkylation but the intensity of each band was less in the CM treated DNA as compared to the untreated control (FIG. 3B). The CMV viral DNA treated with CM was digested in the presence of EcoR1 indicating that CM did not interfere with the enzyme activity of restriction enzymes. It also suggests that centanamycin did not interfere with the protein or enzyme activity of the virus and specifically alkylated the DNA only. However, the CM treated viruses were alkylated efficiently as compared to the control which was observed by the DNA fragmentation during incubation at 50° C. while control untreated viruses have intact DNA (FIG. 3C). That the CM treated viral DNA experienced fragmentation at 50° C. suggests alkylation of the N3 atom in adenine which is thermolabile.

### EXAMPLE 5

### MCMV-Luc Growth Curve Analysis in In Vitro Conditions

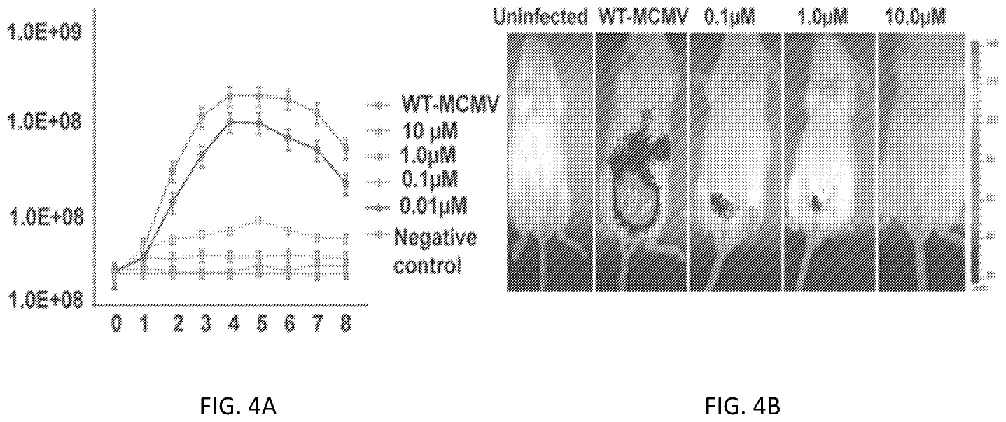

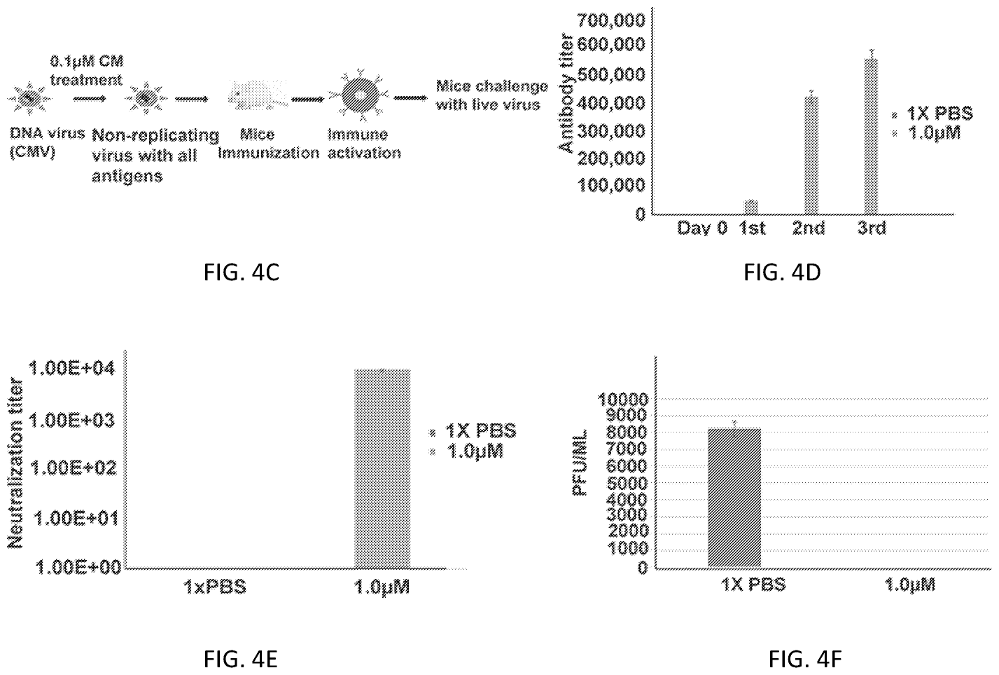

Previous studies in the current disclosure had been conducted in vitro. Thus in order to established an in vivo model of mouse cytomegalovirus (MCMV) study to demonstrate the efficacy of CM in vivo, the growth curve of MCMV was first determined following exposure to different concentrations of CM. This study would identify a CM concentration required to attenuate the MCMV. For this assay, a luciferase-expressing version of MCMV, MCMV-Luc was used, and viral growth was measured as photon counts during intravital imaging using an in vivo imaging system (IVIS). The MCMV-Luc was treated with different concentrations of CM at RT for two hours and then added to NIH 3T3 cells established in 24 well plates in triplicate at a multiplicity of infection (MOI) of 0.1. The MCMV growth curve was determined by measuring the luciferase expression every 24 h using IVIS. The results of this study demonstrated that 1.0 μM of CM allowed the virus to infect host cells but not replicate (FIG. 4A) while 10 μM did not allow the MCMV-Luc to infect the cells and thus show complete inhibition. The MCMV treated with 0.01 μM CM was able to grow and replicate but slower than untreated. These results suggest that 1.0 μM mutate the MCMV so that it can infect the cells but not replicate due to damage DNA. This information was used to perform In vivo analysis of CM effect on the MCMV replication in the mice.

### EXAMPLE 6

### An In Vivo Mouse Model of MCMV Vaccination

The MCMV-Luc virus was purified using a sucrose gradient and titrated. Following purification, 5×10⁵ pfu of MCMV-Luc was treated with 10 μM, 1 μM and 0.1 μM of CM for 2 hours at RT. Thereafter, treated 1×10⁵ pfu were injected intraperitoneally into five mice for each concentration. The results demonstrated that 10 μM CM treated MCMV-Luc did not infect the mice and indicated a complete death of the virus. The mice treated with 0.1 μM and 1 μM CM were able to infect the mice but did not replicate further from the site of infection, indicating a genetically attenuated virus (FIG. 4B). During this attenuated infection, the mouse immune system had sufficient time to recognize the virus as a foreign antigen and generate antibodies specific for the virus. Moreover, since the attenuated MCMV-Luc was whole viral particles, they contained all possible antigens relevant to priming a protective humeral response. As expected, the MCMV-Luc viral particles that were not treated with CM treatment replicated rapidly in the host mice, as observed by IVIS imaging in FIG. 4B.

To determine the potential efficacy of a CM-attenuated MCMV vaccine, five mice per group were vaccinated with 1.0 μM CM treated-MCMV-Luc at 1×10⁶ pfu/mouse with alum adjuvant (illustrated in FIG. 4C). Control mice were injected with sterile PBS. The second and third immunization was performed with a half of the first immunization dose. A total of three immunizations were performed at an interval of two weeks as shown in FIG. 4C and 100 μl of blood was collected 15 days after each injection to determine the virus-specific antibody titer. ELISA results demonstrated that immunized mice had high antibody titer after the second immunization, and the titers further increased following the third immunizations but to a lower extent as compared to earlier injections (FIG. 4D). The serum collected from the immunized mice was used to determine the neutralizing antibody titer and results demonstrated that immunization with CM treated MCMV generates specific neutralization titer (FIG. 4E). Two weeks following the last immunization, mice were rechallenged with MCMV at 10,000 PFU/mouse. Six days following the rechallenge, luciferase assays did not detect the presence of virus infection in immunized mice when compared to control mice. To assess viral replication and infection in the immunized mice, the salivary glands were isolated 10 days post-rechallenge from immunized and control groups. The viral titer was determined in the salivary glands using plaque assays. The results demonstrated that control mice had a high titer value while the immunized group had little to no detectable viral titer in tissue homogenates, indicating that immunized mice had sufficient neutralizing antibodies to suppress and eliminate the virus infection (FIG. 4F).

### EXAMPLE 7

### Selected Discussion

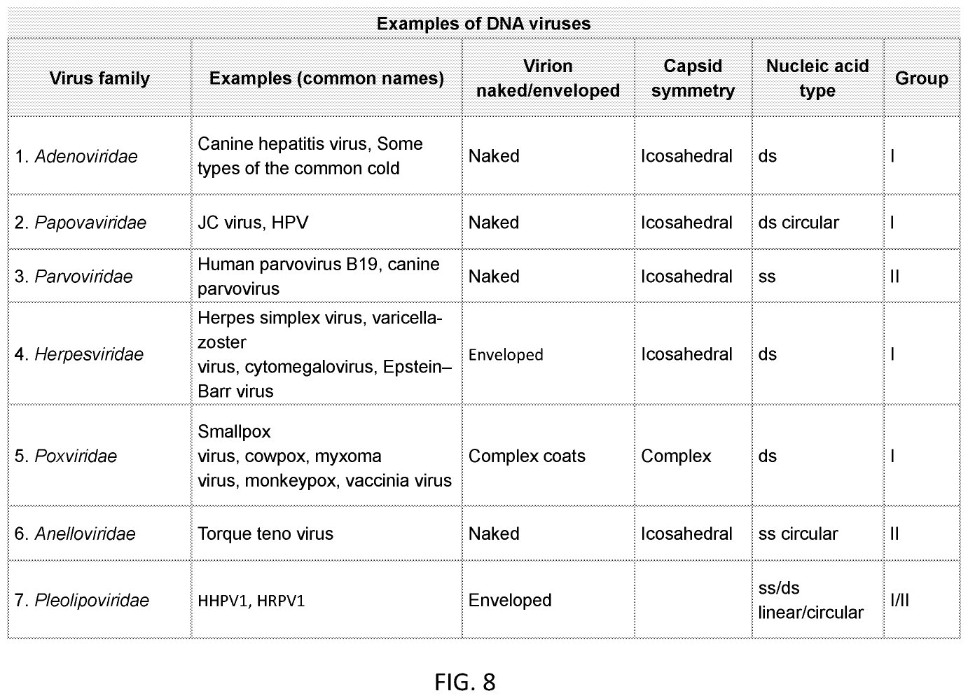

DNA viruses represent a significant threat to global health as well as the economy. In the present disclosure, the effect of CM treatment on virus attenuation was assessed, as was the potential for chemically (CM)-attenuated, live DNA viruses to be used as vaccines (a non-limiting list of possible DNA virus targets are listed in FIG. 8). The replication-defective viruses were able to infect cells without the ability to replicate due to DNA alkylation. These live replication-defective viruses represent an excellent opportunity to present antigens to immune cells and induce productive immune responses. Without wishing to be bound by theory, it can by hypothesized that one of the major advantages of the replication-defective virus vaccine of the present invention is the safety offered by the robust inhibition of virus replication.

In the current study, results demonstrated that CM is an effective antiviral agent that can be used to generate replication-defective live-attenuated DNA viruses. In fact, the results demonstrated that CM readily entered viral particles, alkylated DNA at adenine-N3 in the minor groove of AT-rich sequences, and effectively produced replication-defective live-attenuated viruses. Treatment of HCMV at 0.10 μM of CM for 2 hours at RT produced replication-defective, live attenuated virus. However, 1 μM of CM was needed to produce replication-defective HSV-2. The viruses treated with a higher concentration of CM (100 μM, and 10 μM) are noninfectious, non-replicating dead-virus suggesting an extensive alkylation of viral gDNA. The viruses treated with low concentrations of CM (0.01-0.001 μM) can infect and replicate in the host cells suggesting a low level of viral gDNA alkylation, thus, demonstrating a dose-dependent relationship needed to determine the optimal concentration for specific virus to be attenuated.

Using the MCMV in vivo model, the results presented demonstrated the potential of live, CM-attenuated, replication-defective MCMV for development into a vaccine for subsequent protection and treatment of against infection from wild-type virus. Mice inoculated with the potential vaccine produced a high level of antibody titer sufficient to neutralize live MCMV under in vitro conditions. Moreover, the challenge of immunized mice with live MCMV did not produce an infection and virus load was ˜1000 fold less than unimmunized mice.

In summary, the studies of the present disclosure present evidence that CM-attenuation is an effective method of producing live-attenuated replication-defective DNA viruses that can be used for immunization to produce broadly neutralizing antibodies. While these studies illustrate the ability of CM to attenuate HCMV, MCMV, and HSV and generating live-attenuated replication-defective viruses, future studies will include studies on other DNA viruses and examining any toxicity of the potential vaccines.

### EXAMPLE 8

### Production of Bacterial Vaccines using Centanamycin Treatment

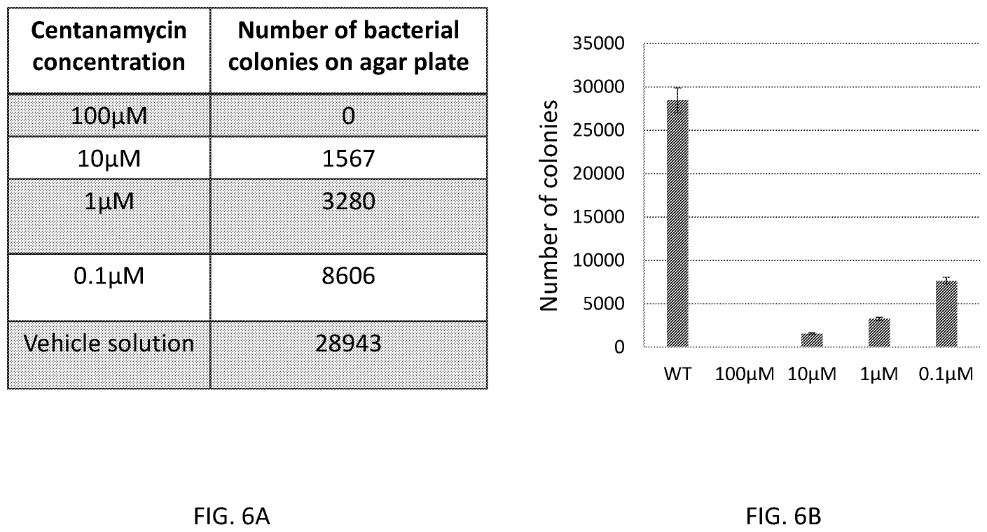

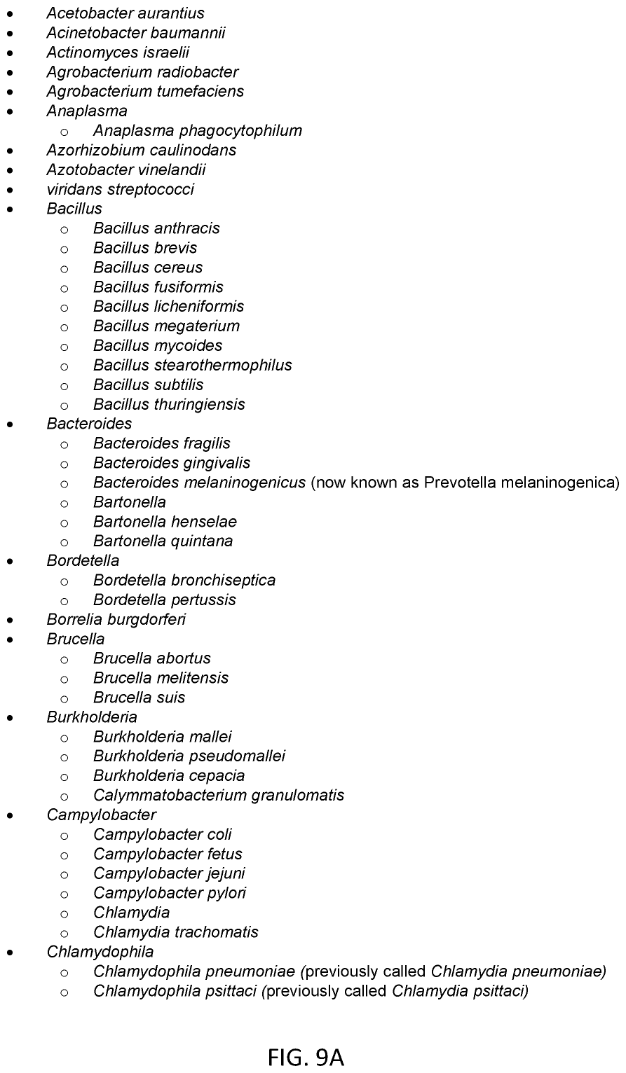

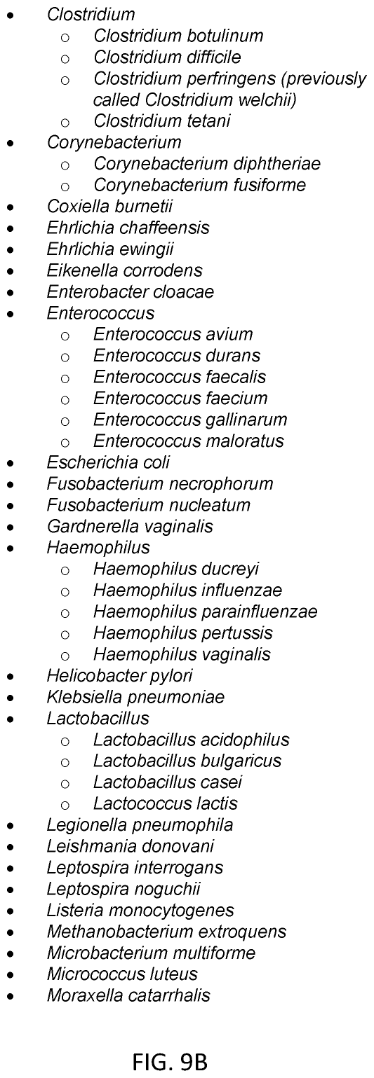

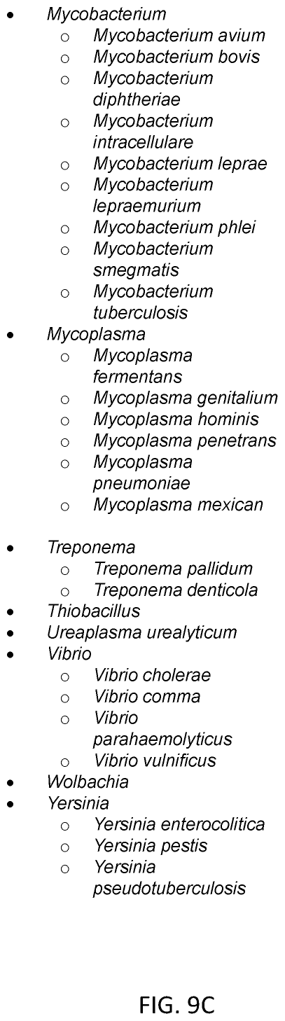

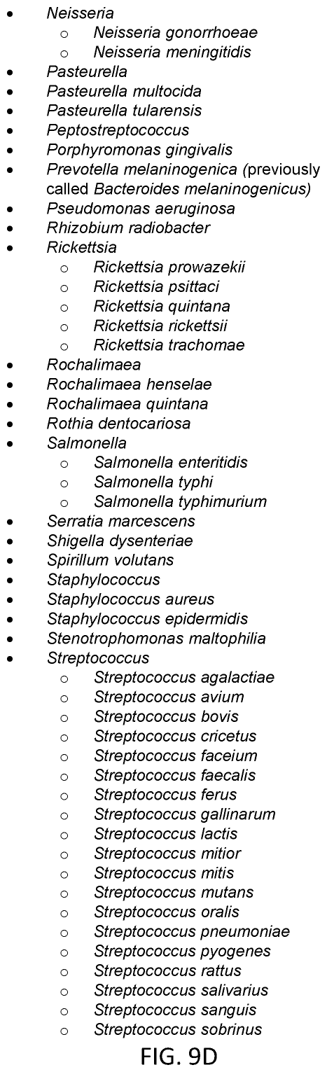

Having demonstrated the ability of centanamycin treatment to produce attenuated and replication-defective viruses for use as vaccines, a series of studies was then undertaken to apply the same strategy to the production of non-replicating bacterial cells for use as immunogenic vaccines (a non-limiting list of potential bacterial vaccine targets are listed in FIGS. 9A-9D). First, a culture of E. coli that had been established by an overnight incubation was diluted 200 times in LB media to make final volume to be 1 ml, followed by treatment with different concentrations of centanamycin (100 μM, 10 μM, 1 μM, and 0.1 μM) for three hours at room temperature. The treated cells were then centrifuged at 10,000 rpm for 1 min, supernatant was removed and the pellet was resuspended in 100 μl of LB and plated on LB agar plates without any antibiotic. The plates were then incubated at 37° C. for 16 hours. To determine the effect of centanamycin, the number of colonies on each plate were counted (FIGS. 6A-6B). The E. coli treated with 100 μM of centanamycin did not grow on the LB agar plate, suggesting that CM treatment at this concentration was toxic. The other concentrations of centanamycin (10 μM, 1 μM and 0.1 μM) were able to reduce the number of bacterial colonies on the agar plate in a dose dependent manner suggesting that centanamycin alkylated the gDNA of the bacteria and reduced growth by mutating the gDNA of the bacteria. Some of these bacteria were replication-defective and attenuated, making them suitable for use as vaccines. These results also confirmed that centanamycin was able to cross the bacterial cell membrane and contact the bacterial gDNA, resulting in chemical modification and mutation.

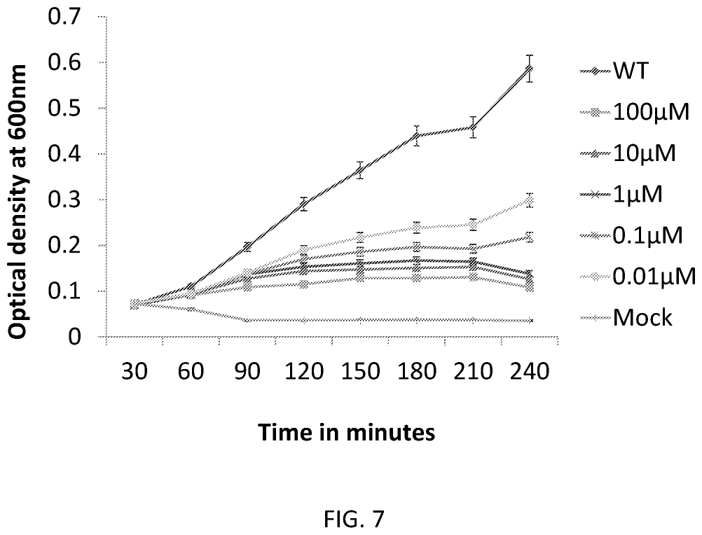

In order to observe the effect of CM on the rate of bacterial growth, a series of studies were conducting observing cultures of treated E. coli (FIG. 7). Here, 10 μl of E. coli culture was added to 100 μl of LB broth and treated with different concentration of CM (100 μM, 10 μM, 1 μM, and 0.1 μM) for two hours at RT. Each treatment condition was repeated in triplicate. The treated E. coli were then added to 96 well round-bottom plates, and cultured at 37° C. in a shaking incubator. The bacterial OD was measured by a Glomax Explorer (Promega) every hour to determine the rate bacterial growth. The results demonstrated that the E. coli treated with 100 μM CM did not grow in the culture. Similarly, E. coli treated with 10 μM CM also did not grow as suggested by the very low optical density (OD) of the bacterial culture. These results suggested that treatment with 100 μM and 10 μM of CM chemically modified the DNA of the bacterial cells. E. coli treated with 0.1 μM and 0.01 μM were able to grow slowly in the culture as evidenced by increasing OD readings over the course of the study. Bacterial cells cultured without prior treatment with CM were used as a control and demonstrated rapid growth in culture, showing a higher OD after 240 minutes suggesting a significantly larger number of the bacteria. Each data point on the graph in FIG. 7 represents the average of three separate measurements.

### EXAMPLE 9

### A Model of Varicella-Zoster Virus (VZV) Attenuation using Centanamycin Treatment

VZV displays strict species specificity. The humanized mouse model represents a practically feasible in vivo model to study VZV pathogenesis and antiviral drug screening. Therefore, the therapeutic efficacy of CM was tested and evaluated in the SCID-hu mouse model.

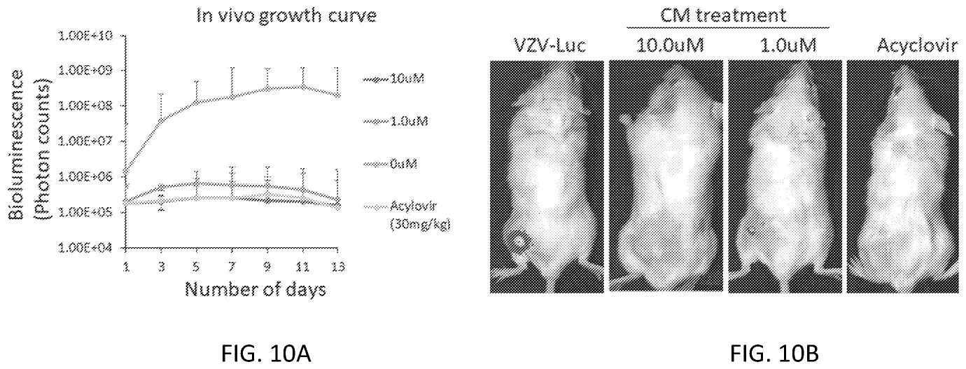

SCID mice of arbitrary sex, 6-8 weeks old were used to produce the SCID-hu mouse model by implanting human fetal skin under the mouse skin as described previously to study the therapeutic efficacy of CM on the VZV replication and attenuation. The human fetal skin tissues were acquired from Advance Biosciences Resources (Alameda, CA) and processed as described previously (Selariu, Anca et al. “ORF7 of varicella-zoster virus is a neurotropic factor.” Journal of virology vol. 86, 16 (2012): 8614-24.). The VZV-Luc was treated with CM of 10.0 μM, and 1.0 μM followed by washing with DMEM media to remove CM. The acyclovir (30 mg/kg) was used as a positive control. VZV-Luc treated with drug vehicle was used as a mock. The viral load was determined every 48 hours as total photon counts in the infected implanted tissues. The total photon counts were determined using IVIS imaging of infected animals as represented in FIGS. 10A and 10B. The image of one mouse from each group is shown in FIG. 10B after 12 days of post-treatment. Four weeks after implantation, the implanted human tissues were surgically exposed and inoculated with 10³ PFU of VZV-Luc. Luciferase activity within the implants was measured every 48 h for 12 days using the IVIS200 imaging system and Live Image software.

In vivo results confirmed that 10.0 μM CM treated VZV did not grow in skin implants (FIGS. 10A and 10B). The VZV treated with 1.0 μM was growing very slowly and have growth defects. The untreated VZV has normal growth and replication in the implanted skin tissues (FIGS. 10A and 10B). The VZV treated with 10.0 μM CM show no luciferase activity in implanted tissues suggesting undetectable viral load whereas mice administered with untreated VZV have luciferase activity (FIGS. 10A & 10B) indicating VZB infection in the implanted tissue.

### Enumerated Embodiments

The following enumerated embodiments are provided, the numbering of which is not to be construed as designating levels of importance.

Embodiment 1 provides a method of producing an immunogenic composition against a pathogen, wherein the pathogen is a virus or bacterium, the method comprising:
a. providing isolated or purified particles and/or cells of the pathogen, or infected cells comprising the pathogen.
b. contacting the pathogenic particles and/or cells, or infected cells comprising the pathogen, with centanamycin, or a salt, solvate, analog, or derivative thereof, thereby rendering the pathogenic particles or cells attenuated and replication-defective, and
c. isolating the attenuated and replication-defective pathogenic particles or cells, thereby producing the immunogenic composition.

Embodiment 2 provides the method of embodiment 1, wherein the pathogen is a virus.

Embodiment 3 provides the method of embodiment 2, wherein the virus is a DNA virus.

Embodiment 4 provides the method of embodiment 3, wherein the DNA virus is selected from the group consisting of adenoviridae, papovaviridae, parvoviridaem, herpesviridae, poxviridae, anelloviridae, pleolipoviridae, and any combination thereof.

Embodiment 5 provides the method of embodiment 1, wherein the pathogen is a bacterium.

Embodiment 6 provides the method of embodiment 5, wherein the bacterium is selected from the group consisting of Escherichia coli, a Mycobacterium, a Streptococcus, a Pseudomonas, a Staphylococcus, a Chlamydia, a Neisseria, Borrelia burgdorferi, a Bruccella, a Listeria, a Legionella, a Shigella, a Campylobacter, a Salmonella, and any combination thereof.

Embodiment 7 provides the method of any one of embodiments 1-6, wherein the pathogen comprises one or more bacteria and/or one or more viruses.

Embodiment 8 provides an immunogenic composition comprising a pharmaceutically acceptable carrier or diluent and an effective amount of isolated or purified particles and/or cells from a pathogen, wherein the pathogen is a virus or bacterium, wherein the isolated or purified pathogenic particles and/or cells are attenuated and replication-defective due to treatment with a compound selected from centanamycin or tafuramycin A, or a salt, solvate, analog, or derivative thereof, and wherein the composition elicits a protective immune response to the pathogenic particles and/or cells in a subject.

Embodiment 9 provides the immunogenic composition of embodiment 8, wherein the compound is a compound of Formula (I):

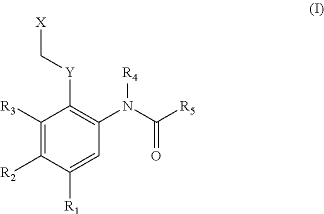

wherein,

R₁ is NHR or OR, where R is selected from H, benzyl, benzyloxycarbonyl, 4-nitrobenzyloxycarbonyl and N′-methylpiperazinyl-N-carbonyl;

R₂ is selected from H and C₁₋₆ alkyl;

R₃ is selected from H and C₁₋₆ alkyl;

or R₂ and R₃, form a fused ring selected from the group consisting of benzene, pyrrole, pyridine, furan, and 5-methylfuran, wherein the fused ring may be optionally substituted with C₁₋₆ alkyl, CF₃, or C₁₋₆ alkyloxycarbonyl;

Y is —CH— to form a five-membered ring with R₄;

X is an electrophilic leaving group;

R₄ is a —CH₂— group bonded to Y to form a five-membered ring; and

R₅ is selected from the group consisting of:

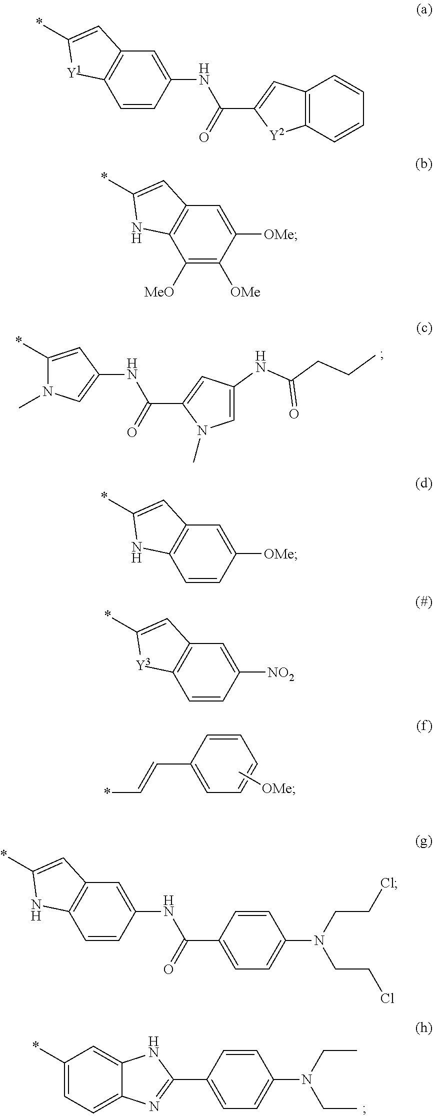

wherein Y¹ and Y² are independently selected from O and NH and wherein Y³ is independently selected from O and NH.

Embodiment 10 provides the immunogenic composition of embodiment 8, wherein the compound is a compound of Formula (II):

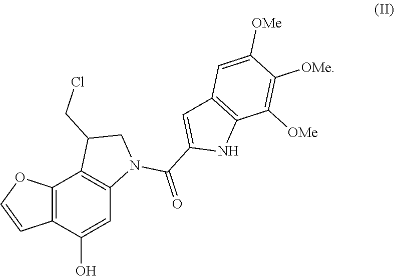 [SMILES: COc1cc2cc(C(=O)N3CC(CCl)c4c3cc(O)c3ccoc43)[nH]c2c(OC)c1OC]

Embodiment 11 provides the immunogenic composition of embodiment 8, wherein the compound is a compound of Formula (III):

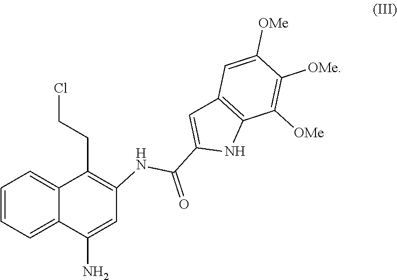 [SMILES: COc1cc2cc(C(=O)Nc3cc(N)c4ccccc4c3CCCl)[nH]c2c(OC)c1OC]

Embodiment 12 provides the immunogenic composition of embodiment 8, wherein the compound is a compound of Formula (IV):

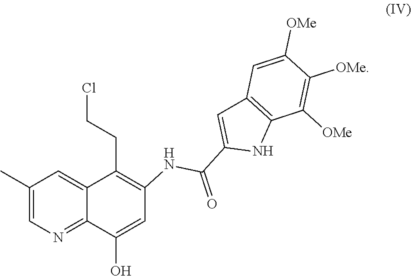 [SMILES: COc1cc2cc(C(=O)Nc3cc(O)c4ncc(C)cc4c3CCCl)[nH]c2c(OC)c1OC]

Embodiment 13 provides the immunogenic composition of embodiment 8, wherein the pathogen is a virus.

Embodiment 14 provides the immunogenic composition of embodiment 13, wherein the virus comprises a DNA virus.

Embodiment 15 provides the immunogenic composition of embodiment 14, wherein the DNA virus is selected from the group consisting of adenoviridae, papovaviridae, parvoviridaem, herpesviridae, poxviridae, anelloviridae, pleolipoviridae, and any combination thereof.

Embodiment 16 provides the immunogenic composition of embodiment 8, wherein the pathogen is a bacterium.

Embodiment 17 provides the immunogenic composition of embodiment 16, wherein the bacterium is selected from the group consisting of Escherichia coli, a Mycobacterium, a Streptococcus, a Pseudomonas, a Staphylococcus, a Chlamydia, a Neisseria, Borrelia burgdorferi, a Bruccella, a Listeria, a Legionella, a Shigella, a Campylobacter, a Salmonella, and any combination thereof.

Embodiment 18 provides the immunogenic composition of any one of embodiments 8-17, wherein the pathogen comprises one or more bacteria and/or one or more viruses.

Embodiment 19 provides a method of stimulating an immune response against a pathogen in a subject, wherein the pathogen is a virus or bacterium, the method comprising administering to the subject an effective amount of the immunogenic composition of any one of embodiments 8-14.

Embodiment 20 provides the method of embodiment 19, wherein the subject is a mammal.

Embodiment 21 provides the method of embodiment 19, wherein the subject is human.

Embodiment 22 provides a method for treating, ameliorating, and/or preventing a pathogen-caused disease in a subject, wherein the pathogen is a virus or bacterium, the method comprising administering to the subject an effective amount of the immunogenic composition of any one of embodiments 8-18.

Embodiment 23 provides the method of embodiment 22, wherein the disease is a viral disease.

Embodiment 24 provides the method of embodiment 23, wherein the viral disease is caused by a DNA virus.

Embodiment 25 provides the method of embodiment 24, wherein the DNA virus is selected from the group consisting of adenoviridae, papovaviridae, parvoviridaem, herpesviridae, poxviridae, anelloviridae, pleolipoviridae, and any combination thereof.

Embodiment 26 provides the method of embodiment 22, wherein the disease is a bacterial disease.

Embodiment 27 provides the method of embodiment 26, wherein the bacterial disease is caused by a bacterium selected from the group consisting of Escherichia coli, a Mycobacterium, a Streptococcus, a Pseudomonas, a Staphylococcus, a Chlamydia, a Neisseria, Borrelia burgdorferi, a Bruccella, a Listeria, a Legionella, a Shigella, a Campylobacter, a Salmonella, and any combination thereof.

Embodiment 28 provides the method of any one of embodiments 19-27, wherein the subject is a mammal.

Embodiment 29 provides the method of any one of embodiments 19-27, wherein the subject is human.

### Other Embodiments

The recitation of a listing of elements in any definition of a variable herein includes definitions of that variable as any single element or combination (or sub combination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A method of producing an immunogenic composition against a pathogen, wherein the pathogen is a virus or bacterium, the method comprising:
a. providing isolated or purified particles or cells of the pathogen, or infected cells comprising the pathogen.
b. contacting the pathogenic particles or cells, or infected cells comprising the pathogen, with centanamycin, or a salt, solvate, analog, or derivative thereof, thereby rendering the pathogenic particles or cells attenuated and replication-defective, and
c. isolating the attenuated and replication-defective pathogenic particles or cells, thereby producing the immunogenic composition.

2. The method of claim 1, wherein the pathogen comprises a virus or a bacterium.

3. The method of claim 2, wherein the pathogen comprises the virus, and wherein the virus is a DNA virus.

4. The method of claim 3, wherein the DNA virus is selected from the group consisting of adenoviridae, papovaviridae, parvoviridaem, herpesviridae, poxviridae, anelloviridae, pleolipoviridae, and any combination thereof.

5. (canceled)

6. The method of claim 2, wherein the pathogen comprises the bacterium, and wherein the bacterium is selected from the group consisting of Escherichia coli, a Mycobacterium, a Streptococcus, a Pseudomonas, a Staphylococcus, a Chlamydia, a Neisseria, Borrelia burgdorferi, a Bruccella, a Listeria, a Legionella, a Shigella, a Campylobacter, a Salmonella, and any combination thereof.

7. (canceled)

8. An immunogenic composition comprising a pharmaceutically acceptable carrier or diluent and an effective amount of isolated or purified particles and/or cells from a pathogen,
wherein the pathogen is a virus or bacterium,
wherein the isolated or purified pathogenic particles or cells are attenuated and replication-defective due to treatment with a compound selected from centanamycin or tafuramycin A, or a salt, solvate, analog, or derivative thereof, and
wherein the composition elicits a protective immune response to the pathogenic particles or cells in a subject.

9. The immunogenic composition of claim 8, wherein the compound is a compound of Formula (I): 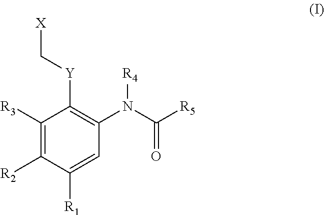
wherein,
R₁ is NHR or OR, where R is selected from H, benzyl, benzyloxycarbonyl, 4-nitrobenzyloxycarbonyl and N′-methylpiperazinyl-N-carbonyl;
R₂ is selected from H and C₁₋₆ alkyl;
R₃ is selected from H and C₁₋₆ alkyl;
or R₂ and R₃, form a fused ring selected from the group consisting of benzene, pyrrole, pyridine, furan, and 5-methylfuran, wherein the fused ring may be optionally substituted with C₁₋₆ alkyl, CF₃, or C₁₋₆ alkyloxycarbonyl;
Y is —CH— to form a five-membered ring with R₄;
X is an electrophilic leaving group;
R₄ is a —CH₂— group bonded to Y to form a five-membered ring; and
R₅ is selected from the group consisting of: 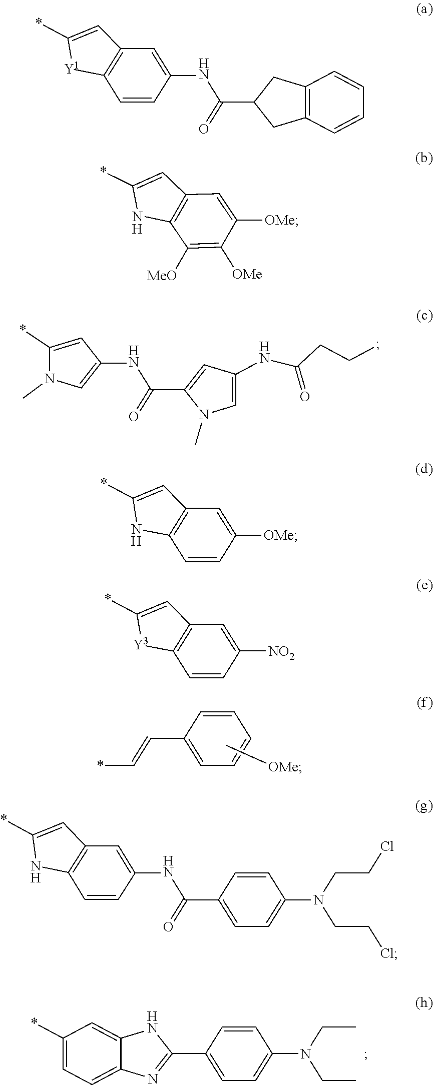
wherein Y¹ and Y² are independently selected from O and NH and wherein Y³ is independently selected from O and NH.

10. The immunogenic composition of claim 8, wherein at least one of the following applies:
(a) the compound is a compound of Formula (II): 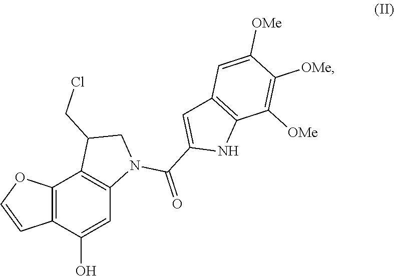 [SMILES: COc1cc2cc(C(=O)N3CC(CCl)c4c3cc(O)c3ccoc43)[nH]c2c(OC)c1OC]
(b) the compound is a compound of Formula (III): 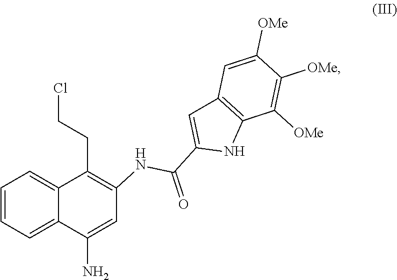 [SMILES: COc1cc2cc(C(=O)Nc3cc(N)c4ccccc4c3CCCl)[nH]c2c(OC)c1OC]
(c) the compound is a compound of Formula (IV):

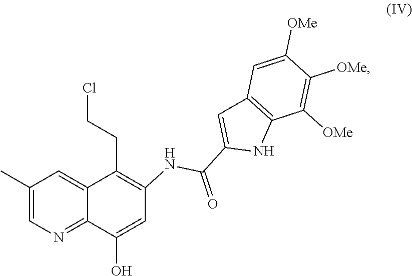 [SMILES: COc1cc2cc(C(=O)Nc3cc(O)c4ncc(C)cc4c3CCCl)[nH]c2c(OC)c1OC]

11. (canceled)

12. (canceled)

13. The immunogenic composition of claim 8, wherein the pathogen comprises a virus or a bacterium.

14. The immunogenic composition of claim 13, wherein the pathogen comprises the virus, and wherein the virus comprises a DNA virus.

15. The immunogenic composition of claim 14, wherein the DNA virus is selected from the group consisting of adenoviridae, papovaviridae, parvoviridaem, herpesviridae, poxviridae, anelloviridae, pleolipoviridae, and any combination thereof.

16. (canceled)

17. The immunogenic composition of claim 13, wherein the pathogen comprises the bacterium, and the bacterium is selected from the group consisting of Escherichia coli, a Mycobacterium, a Streptococcus, a Pseudomonas, a Staphylococcus, a Chlamydia, a Neisseria, Borrelia burgdorferi, a Bruccella, a Listeria, a Legionella, a Shigella, a Campylobacter, a Salmonella, and any combination thereof.

18. (canceled)

19. A method of stimulating an immune response against a pathogen in a subject, wherein the pathogen is a virus or bacterium, the method comprising administering to the subject an effective amount of the immunogenic composition of claim 8.

20. The method of claim 19, wherein the subject is a mammal, optionally wherein the mammal is human.

21. (canceled)

22. A method for treating, ameliorating, and/or preventing a pathogen-caused disease in a subject, wherein the pathogen is a virus or bacterium, the method comprising administering to the subject an effective amount of the immunogenic composition of claim 8.

23. The method of claim 22, wherein the disease is a viral disease or a bacterial disease.

24. The method of claim 23, wherein the disease is the viral disease, and wherein the viral disease is caused by a DNA virus.

25. The method of claim 24, wherein the DNA virus is selected from the group consisting of adenoviridae, papovaviridae, parvoviridaem, herpesviridae, poxviridae, anelloviridae, pleolipoviridae, and any combination thereof.

26. (canceled)

27. The method of claim 22, wherein the disease is a bacterial disease, and wherein the bacterial disease is caused by a bacterium selected from the group consisting of Escherichia coli, a Mycobacterium, a Streptococcus, a Pseudomonas, a Staphylococcus, a Chlamydia, a Neisseria, Borrelia burgdorferi, a Bruccella, a Listeria, a Legionella, a Shigella, a Campylobacter, a Salmonella, and any combination thereof.

28. The method of claim 19, wherein the subject is a mammal, optionally wherein the mammal is a human.

29. (canceled)
